SEQUENCE LISTING (2 sequences)

SEQ ID NO: 1 - protein, 5 aa, Artificial Sequence
  sequence is completely synthesized
TFLLR

SEQ ID NO: 2 - protein, 5 aa, Artificial Sequence
  sequence is completely synthesized
FTLLR

(19) United States
(12) Patent Application Publication
(10) Pub. No.: US 2014/0378502 A1
(43) Pub. Date: Dec. 25, 2014

(54) Par1 Inhibitors for Use in the Treatment or Prevention of Paramyxoviridae Infections

(71) Applicants: Béatrice Riteau, Pollyonnais (FR); Guy Boivin, Quebec (CA); Laetitia Aerts, Quebec (CA); Marie-Eve Hamelin, Quebec (CA)

(72) Inventors: Béatrice Riteau, Pollyonnais (FR); Guy Boivin, Quebec (CA); Laetitia Aerts, Quebec (CA); Marie-Eve Hamelin, Quebec (CA)

(73) Assignees: Université Laval, Quebec (CA); Institut National de la Recherche Agronomique, Paris (FR)

(21) Appl. No.: 14/117,309

(22) PCT Filed: May 11, 2012
(86) PCT No.: PCT/CA2012/000455

§ 371 Date: Sep. 5, 2014

Related U.S. Application Data
(60) Provisional application No. 61/485,419, filed on May 12, 2011.

(51) Int. Cl. A61K 31/4745; A61K 45/06

(52) U.S. Cl. CPC A61K 31/4745; A61K 45/06
USPC 514/292

(57) ABSTRACT

The present invention is concerned with the use of Protease-Activated Receptor-1 (PAR1) inhibitors for preventing or treating a Paramyxoviridae infection in a subject. Described herein are methods, compounds and pharmaceutical compositions useful in addressing such infections, and more particularly infections from human respiratory syncytial virus (hRSV) and human metapneumovirus (hMPV).

### FIELD OF THE INVENTION

The invention relates to the field of virology. More particularly, it relates to the prevention and/or treatment of Paramyxoviridae infections, including infections from human respiratory syncytial virus (hRSV) or human metapneumovirus (hMPV), by using Protease-Activated Receptor-1 (PAR1) inhibitors.

### BACKGROUND OF THE INVENTION

Acute respiratory tract infections (ARTI) are a leading cause of morbidity and the second most important cause of death throughout the world among children <5 years old. The majority of ARTI are caused by viruses, among which respiratory syncytial virus (RSV) and the closely related human metapneumovirus (hMPV) figure prominently. Although RSV is the most important etiologic agent of bronchiolitis and pneumonia in infants and young children, hMPV has been consistently reported as the second or third most important cause of bronchiolitis and hospitalization for any ARTI.

Human metapneumovirus (hMPV) belongs to the Metapneumovirus genus within the Pneumovirinae subfamily of the Paramyxoviridae family. Human respiratory syncytial virus (hRSV), the most closely related human pathogen belongs to a separate genus (Pneumovirus) within the same subfamily (Wyde P. R., Antiviral research 39 (1998), 63-79). The hMPV genome consists of a single negative strand of RNA of approximately 13 Kb in length containing 8 genes that code for 9 proteins. Human MPV is a ubiquitous virus producing yearly epidemics in temperate countries that usually peak in late winter to early spring, coincident with or slightly later than hRSV. One UK study estimated an annual hMPV hospitalization rate of 3.2 per 1000 children whereas it was 1.2/1000 (27000 hospitalizations/year) compared to 3/1000 for RSV in a US study. The clinical manifestations of hMPV are indistinguishable from those of RSV; however, some studies have observed more severe disease associated with RSV, as evidenced by higher disease severity scores, longer duration of symptoms, more frequent requirements for oxygen, respiratory support or ICU admission. More specifically, diagnoses of URTI (with or without fever), acute otitis media and bronchiolitis with or without pneumonia have been most commonly reported in the pediatric population.

There are still no specific treatments or vaccines approved for hMPV. Ribavirin, a nucleoside analogue currently approved for use to treat and prevent RSV infections, has good in vitro activity against hMPV and was effective in a mouse model of hMPV infection and in a lung transplant recipient with hMPV pneumonia. Although exhibiting good inhibitory activity against hMPV in vitro, the clinical benefit of intravenous immunoglobulins remains unclear. In addition, there was minimal benefit for adjunctive corticosterone treatment in hMPV-infected mice. Considering the limitations associated with ribavirin (teratogenicity and myelosuppressive side-effects) and adjunctive therapy, development of new therapeutic modalities for hMPV and hRSV is of high importance.

Activation of host innate immune system aims at controlling the spreading and deleterious effects of Paramyxoviridae infection. However, excessive inflammatory response, due to a dysregulation of cytokine release and strong recruitment of neutrophils at the site of infection, mediate severe lung inflammation and increased pathogenesis of viruses of the Paramyxoviridae family. The sites of virus replication in the respiratory tract represent complex microenvironments, in which extracellular proteases are present in large amounts. Some of these proteases (trypsin, tryptase) can play a role both in virus replication and innate immune responses as they are important mediators of inflammatory processes through the activation of a family of receptors called Protease-Activated Receptors (PARs).

PARs are G protein coupled receptors found on the surface of cells from a wide variety of tissues. To date four PARs, activated by different proteases, have been cloned (PAR1-4) and PAR agonists and PAR antagonists are known (Adams N. M. et al., Structure, function and pathophysiology of protease activated receptors, Pharmacology & Therapeutics (2011), doi:10.1016/jpharmthera.2011.01.003; Lee H. and Hamilton J. R., Physiology, pharmacology, and therapeutic potential of protease-activated receptors in vascular disease, Pharmacology & Therapeutics (2012), doi:10.1016/j.pharmthera.2012.01.007). Although it has been 20 years since the discovery of PAR and that agonists and antagonists of PAR are known, until now, the role of PAR1 in lung human metapneumovirus or respiratory syncytial virus infection has never been documented and the specific role for PAR1 activation/inactivation in vivo or in vitro has never been addressed. Prior to the present invention, it had not been suggested that PAR1 inhibitors could find a use in preventing, treating, improving, and/or alleviating Paramyxoviridae infections. The present invention is different from International PCT publication WO 2011/058183 which teaches the use of PAR1 antagonists for the treatment or prevention of influenza virus type A infections, influenza virus type A being a genus of the Orthomyxoviridae family.

There is thus a need for the discovery and development of novel antivirals/immunomodulators. There is more particularly a need for treatment against human metapneumovirus (hMPV), and human respiratory syncytial virus (hRSV).

### SUMMARY OF THE INVENTION

According to one aspect, the invention concerns a method for preventing or treating a Paramyxoviridae infection in a subject, the method comprising administering to the subject a Protease-Activated Receptor-1 (PAR1) inhibitor.

According to another aspect, the invention concerns a method for the prevention or treatment of Paramyxoviridae infection in a human subject (e.g. a Pneumovirinae infection), the method comprising administering to the subject a PAR1 inhibitor before infection or at any time after infection (preferably shortly) or shortly after appearance of symptoms of infection, e.g. within one day, within two days or within three days.

According to a further aspect, the invention concerns the use of a PAR1 inhibitor for the manufacture of a medicine for the prevention or treatment of a Paramyxoviridae infection (preferably a Pneumovirinae infection) in a human subject.

According to a further aspect, the invention concerns a pharmaceutical composition for the prevention or treatment of a Paramyxoviridae infection (preferably a Pneumovirinae infection) in a subject, the composition comprising a PAR1 inhibitor and a pharmaceutically acceptable carrier.

Another related aspect of the invention concerns an antiviral composition comprising a PAR1 inhibitor in combination with a neuraminidase inhibitor.

Another related aspect of the invention concerns a medicine (e.g. an antiviral composition) comprising a PAR1 inhibitor in combination with a drug selected from the group consisting of ribavirin, peginterferon alfa-2b, peginterferon alfa-2a, an antibiotics, and anti-inflammatory compounds such as corticosteroids.

Additional aspects, advantages and features of the present invention will become more fully understood from the detailed description given herein and from the accompanying drawings, which are exemplary and should not be interpreted as limiting the scope of the invention.

### BRIEF DESCRIPTION OF THE FIGURES

FIG. 1 is a line graph showing weight loss in hMPV-infected mice treated with PAR-1 agonist for 3 days according to the Examples. Briefly, four groups of 6 mice were infected intranasally with hMPV (4-6×10⁵ TCID₅₀) or mock infected and simultaneously treated with 50 () or 500 μM (▪) of PAR1 agonists (TFLLR-NH2) for 3 days. The mice were followed for weight loss and mortality for 14 days. The treated mice show an increase in weight loss compared to infected, untreated mice (□) after hMPV infection and an induction of mortality was observed after PAR1 treatment but not in untreated mice. Arrow indicates when mice reached human endpoint (full line: 50 μM, dotted line: 500 μM); number indicates number of mice that reached human endpoint; * indicate a significant difference in weight loss between mice treated with PAR1 agonist (500 μM) and untreated mice (* p<0.05) as determined by Repeated Measures (ANOVA). (o) represent uninfected, untreated mice.

FIG. 2 is a line graph showing weight loss in hMPV-infected mice treated with PAR-1 antagonist for 3 days according to the Examples. Briefly, four groups of 6 mice were infected intranasally with hMPV (4-6×10⁵ TCID₅₀) or mock infected and simultaneously treated with 50 () or 500 μM (▪) of PAR1 antagonists (SCH-79797: N3-cyclopropyl-7-{[4(I-methylethyl)phenyl]methyl)-7H-pyrrolo[3,2-f]quinazoline-I,3-diamine) for 3 days. The mice were followed for weight loss and mortality for 14 days. Mice treated with PAR1 antagonists have reduced weight loss compared to infected and untreated mice after hMPV infection. † indicate a significant difference in weight loss between mice treated with PAR1 antagonist (50 μM) and untreated infected mice (□) (†p<0.05; ††p<0.01); * indicate a significant difference in weight loss between mice treated with PAR1 antagonist (500 μM) and untreated infected mice (* p<0.05; **p<0.01; *** p<0.001). (o) represent uninfected, untreated mice.

FIG. 3 is a bar graph showing lung viral titers in hMPV-infected mice treated with PAR-1 agonist or antagonist for 3 days according to the Examples. Briefly, six mice per group were infected intranasally with hMPV (4-6×10⁵ TCID₅₀) or mock infected and simultaneously treated with 50 or 500 μM of PAR1 agonists (TFLLR-NH2) or PAR1 antagonists (SCH-79797: N3-cyclopropyl-7-{[4(I-methylethyl)phenyl]methyl)-7H-pyrrolo[3,2-f]quinazoline-I,3-diamine) for 3 days. The mice were sacrificed on day 5 post infection and lung viral titers were determined. * indicate a significant difference in lung viral titers between mice treated with PAR1 antagonist and infected/untreated control (0 μM) mice (* p<0.05, ** p<0.01), which was not the case with treatment with PAR-1 agonist.

FIGS. 4A-4D are bar graphs showing lung cytokines in hMPV-infected mice treated with PAR-1 agonist or antagonist for 3 days. Briefly, six mice per group were infected intranasally with hMPV (4-6×10⁵ TCID₅₀) or mock infected and simultaneously treated with 50 or 500 μM of PAR1 agonists (TFLLR-NH2) or PAR1 antagonists (SCH-79797: N3-cyclopropyl-7-{[4(I-methylethyl)phenyl]methyl)-7H-pyrrolo[3,2-f]quinazoline-I,3-diamine) for 3 days. The mice were sacrificed on day 5 post infection and cytokine levels were assessed using Luminex™ (Bio-plex™ assay from Bio-Rad). Levels of IL-6 (FIG. 4A), IL-12 (p40) (FIG. 4B), IL-12 (p70) (FIG. 4C) and MCP-1 (FIG. 4D) were generally more elevated following treatment with PAR1 agonists compared to their respective control (0 uM) whereas they remained stable following treatment with PAR1 antagonists (* p<0.05; **p<0.01).

FIG. 5 is a line graph showing weight loss in hMPV-infected mice treated with PAR-1 agonist for 5 days according to the Examples. Briefly, four groups of 6 mice were infected intranasally with hMPV (6-8×10⁵ TCID₅₀) or mock infected and simultaneously treated with 500 μM of PAR1 agonists (TFLLR-NH2) or H₂O for 5 days. The mice were followed for weight loss and mortality for 14 days. Uninfected/treated () and uninfected/untreated (o) mice did not lose weight or showed any clinical signs. The treated/infected mice (▪) showed comparable weight loss to infected/untreated mice (□).

FIG. 6 is a line graph showing weight loss in hMPV-infected mice treated with PAR-1 antagonist for 5 days according to the Examples. Briefly, four groups of 6 mice were infected intranasally with hMPV (6-8×10⁵ TCID₅₀) or mock infected and simultaneously treated with 500 μM of PAR1 antagonists (SCH-79797: N3-cyclopropyl-7-{[4(I-methylethyl)phenyl]methyl)-7H-pyrrolo[3,2-f]quinazoline-I,3-diamine) or DMSO for 5 days. The mice were followed for weight loss and mortality for 14 days. Uninfected/treated () and uninfected/untreated (o) mice did not lose weight or showed any clinical signs. The treated/infected mice (▪) were protected from weight loss. * indicate a significant difference in weight loss between infected mice treated with PAR1 antagonist and untreated/infected mice (□) (* p<0.05; *** p<0.001).

FIG. 7 is a line graph showing weight loss in hMPV-infected mice treated with an unrelated peptide for 5 days according to the Examples. Briefly, four groups of 6 mice were infected intranasally with hMPV (6-8×10⁵ TCID₅₀) or mock infected and simultaneously treated with 500 μM of the control (negative) peptide that does not affect PAR-1 (FTLLR-NH2) or H₂O for 5 days. The mice were followed for weight loss and mortality for 14 days. Uninfected/treated () and uninfected/untreated (o) mice did not lose weight or show any clinical signs. The treated/infected mice (▪) show comparable weight loss to infected/untreated mice (□).

FIG. 8 is a bar graph representing lung viral titers in hMPV-infected mice treated with PAR-1 agonist or antagonist for 5 days according to the Examples. Briefly, groups of 6 mice were infected intranasally with hMPV (6-8×10⁵ TCID₅₀) and simultaneously treated for 5 days with 500 μM of PAR1 agonist (TFLLR-NH2), PAR1 antagonist (SCH-79797: N3-cyclopropyl-7-{[4(I-methylethyl)phenyl]methyl)-7H-pyrrolo[3,2-f]quinazoline-I,3-diamine) or the control (negative) peptide (FTLLR-NH2). Control groups were given H₂O or DMSO. The mice were sacrificed on day 5 post infection and lung viral titers were determined by inoculating 10-fold serial dilutions of virus into 24-well plates containing LLC-MK2 cells. * indicate a significant difference in viral titers between mice treated with PAR1 antagonist and all other groups (* p<0.05, ** p<0.01).

FIGS. 9A-9B are bar graphs representing pulmonary cytokine/chemokine levels in hMPV-infected mice treated with PAR-1 agonist or antagonist for 5 days according to the Examples. Briefly, groups of 6 mice were infected intranasally with hMPV (6-8×10⁵ TCID₅₀) and simultaneously treated for 5 days with 500 μM of PAR1 agonist (TFLLR-NH2), PAR1 antagonist (SCH-79797: N3-cyclopropyl-7-{[4(I-methylethyl)phenyl]methyl)-7H-pyrrolo[3,2-f]quinazoline-I,3-diamine) or the control (negative) peptide (FTLLR-NH2). The mice were sacrificed on day 5 post infection and pulmonary cytokine/chemokine levels were determined by Luminex™ (Millipore). Results for IFN-γ, IL-4, IL-6 and IL-12(p40) are represented in FIG. 9A, results for KC, MCP-1, MIP-1a and RANTES are represented in FIG. 9B. In both figures, infected/treated mice are represented by white bars, uninfected mice by black bars. Control groups (no treatment in FIGS. 9A and 9B) were infected and given H₂O (grey bars) or DMSO (white bars) or were uninfected and H₂O treated (black bars). * indicate a significant reduction in pulmonary cytokine/chemokine levels between mice treated with PAR1 antagonist and all other groups (* p<0.05, ** p<0.01, *** p<0.001) [symbol] indicate a significant increase in pulmonary cytokine/chemokine levels between mice treated with PAR1 agonist and all other groups ( [symbol] p<0.05, [symbol] [symbol] <0.01, [symbol] [symbol] [symbol] p<0.001)

FIGS. 10A-10C are bar graphs representing lung histopathological scores in hMPV-infected mice treated with PAR-1 agonist or antagonist for 5 days according to the Examples. Briefly, groups of 6 mice were infected intranasally with hMPV (6-8×10⁵ TCID₅₀) and simultaneously treated for 5 days with 500 μM of PAR1 agonist (TFLLR-NH2) (FIG. 10A), PAR1 antagonist (SCH-79797: N3-cyclopropyl-7-{[4(I-methylethyl)phenyl]methyl)-7H-pyrrolo[3,2-f]quinazoline-I,3-diamine) (FIG. 10B) or the control (negative) peptide (FTLLR-NH2) (FIG. 10C). Control groups were given H₂O or DMSO. The mice were sacrificed on day 5 post infection and lungs were analysed for histopathology (a: bronchial/endobronchial inflammation; b: peribronchial inflammation; c: perivascular inflammation; d: interstitial inflammation; e: pleural inflammation; f: intra-alveolar inflammation). Uninfected/untreated mice (grey bars), uninfected/treated mice (lined bars), infected/untreated mice (white bars) and infected/treated mice (black bars) were compared using two-way ANOVA. * indicate a significant difference in histopathological score (* p<0.05, ** p<0.01, *** p<0.001).

FIG. 11 is a panel showing pictures of lung inflammation in hMPV-infected mice treated with PAR-1 agonist or antagonist for 5 days according to the Examples. Briefly, groups of 6 mice were infected intranasally with hMPV (6-8×10⁵ TCID₅₀) and simultaneously treated for 5 days with 500 μM of PAR1 agonist (TFLLR-NH2), PAR1 antagonist (SCH-79797: N3-cyclopropyl-7-{[4(I-methylethyl)phenyl]methyl)-7H-pyrrolo[3,2-f]quinazoline-I,3-diamine) or the control peptide (FTLLR-NH2). Control groups were given H₂O or DMSO (only DMSO is represented here). The mice were sacrificed on day 5 post infection and lungs were removed. Digitalized images were obtained from formalin-fixed paraffin-embedded hematoxylin-eosin stained histologic sections of lung tissue scanned at 20× with a Nanozoomer™ (Hamamatsu) and viewed with ImageScope™ software (Aperio). Microphotographs were extracted from representative areas on the digitalized slide images. Lung inflammation is observed after infection in all groups except the one treated with the PAR-1 antagonist compound.

FIG. 12 is a line graph showing weight loss in hMPV-infected mice treated with PAR-1 agonist or PAR-1 antagonist for 5 days according to the Examples. Briefly, four groups of 6 mice were infected intranasally with hMPV (6-8×10⁵ TCID₅₀) or mock infected and simultaneously treated with 500 μM of PAR1 agonists (TFLLR-NH2), PAR1 antagonists (SCH-79797: N3-cyclopropyl-7-{[4(I-methylethyl)phenyl]methyl)-7H-pyrrolo[3,2-f]quinazoline-I,3-diamine) or H₂O for 5 days. The mice were followed for weight loss and mortality for 14 days. Infected/PAR-1 antagonist treated () and uninfected/untreated (o) mice did not lose weight or show any clinical signs. The PAR-1 agonist treated/infected mice (▪) showed comparable or increased weight loss compared to infected/untreated mice (□).

FIG. 13 is a line graph showing weight loss in hMPV-infected mice treated with PAR-1 agonist or PAR-1 antagonist for 5 days post-exposure, according to the Examples. Briefly, four groups of 6 mice were infected intranasally with hMPV (6-8×10⁵ TCID₅₀) or mock infected and treated, 24 h post infection, with 500 μM of PAR1 agonists (TFLLR-NH2), PAR1 antagonists (SCH-79797: N3-cyclopropyl-7-{[4(I-methylethyl)phenyl]methyl)-7H-pyrrolo[3,2-f]quinazoline-I,3-diamine) or H₂O for 5 days. Infected/PAR1 antagonists treated () started losing weight later and lost less weight than untreated infected mice, although the difference was not found to be statistically significant. The PAR1 agonist treated/infected mice (▪) showed more weight loss than infected/untreated mice (□), and regained weight more slowly. [symbol] [symbol] [symbol] indicate a significant difference in weight loss between infected mice treated with PAR1 agonist and untreated/infected mice ( [symbol] p<0.05; [symbol] [symbol] [symbol] p<0.001). (o) indicates the uninfected/untreated controls.

FIG. 14 is a bar graph representing lung viral titers in hMPV-infected mice treated with PAR-1 agonist or antagonist for 5 days according to the Examples. Briefly, groups of 6 mice were infected intranasally with hMPV (4-6×10⁵ TCID50) and were treated for 5 days either simultaneously (white bars) or 24 h post infection (gray bars) with 500 μM of PAR1 agonist (TFLLR-NH2), PAR1 antagonist (SCH-79797: N3-cyclopropyl-7-{[4-(I-methylethyl)phenyl]methyl)-7H-pyrrolo[3,2-f]quinazoline-I,3-diamine) or were left untreated (black bars). The mice were sacrificed on day 5 post infection and lung viral titers were determined by inoculating 10-fold serial dilutions of virus into 24-well plates containing LLC-MK2 cells. * indicate a significant difference in viral titers between mice treated with PAR1 antagonist and infected/untreated mice (* p<0.05).

FIG. 15 is a bar graph representing lung viral titers in hRSV-infected mice treated with PAR-1 agonist or antagonist for 5 days according to the Examples. Briefly, groups of 6 mice were infected intranasally with hRSV (2×10⁵ TCID50) and were treated simultaneously with 500 μM of PAR1 agonist (TFLLR-NH2) (grey bars), PAR1 antagonist (SCH-79797: N3-cyclopropyl-7-{[4-(I-methylethyl)phenyl]methyl)-7H-pyrrolo[3,2-f]quinazoline-I,3-diamine) (black bars) or were left untreated (white bars). The mice were sacrificed on day 5 post infection and lung viral titers were determined by inoculating 10-fold serial dilutions of virus into 24-well plates containing Hep2 cells. * indicate a significant difference in viral titers between mice treated with PAR1 antagonist and all other groups (* p<0.05, ** p<0.01).

### DETAILED DESCRIPTION OF THE INVENTION

### A) General Overview of the Invention

The inventors have discovered that Protease-Activated Receptor-1 (PAR1) plays a role in Paramyxoviridae infections. The inventors have also demonstrated that PAR1 inhibitors find a use in preventing, treating, improving, and/or alleviating such viral infections. These unexpected findings open new avenues of prevention and treatment of virus infections. Accordingly, the invention concerns methods and compositions for preventing and/or treating Paramyxoviridae infections in a subject.

### B) Pharmaceutical Applications

In one of its broadest aspect, the invention is concerned with methods and compositions for preventing or treating Paramyxoviridae infections in a subject. In embodiments, the method comprises administering to the subject a Protease-Activated Receptor-1 (PAR1) inhibitor (e.g., antagonist).

In embodiments, prevention or treatment against a Paramyxoviridae infection may comprise determining before, during and after administration of the PAR1 inhibitor the presence or titer of viruses.

The term “subject” includes living organisms in which a Paramyxoviridae infection can occur. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as wild and domestic bird species (e.g. chickens), and transgenic species thereof. Preferably, the subject is a mammal in need of prevention or treatment against a Paramyxoviridae infection. More preferably, the subject is a human.

As used herein, the term “Paramyxoviridae infection” refers to any infection caused by a virus member of the family Paramyxoviridae. The family Paramyxoviridae is composed of a diverse group of viruses and is divided into two subfamilies, Paramyxovirinae and Pneumovirinae. A number of important human diseases are caused by these viruses. These include mumps and measles (caused by viruses from the Paramyxovirinae subfamily). It also includes bronchiolitis and/or pneumonia, especially in children, caused by the respiratory syncytial virus (RSV) and by the human metapneumovirus which belong to the Pneumovirinae subfamily. In some embodiments, the Paramyxoviridae infection is an infection by a virus of the Subfamily Pneumovirinae, e.g. a virus from the genus pneumovirus or from the genus metapneumovirus. In some embodiments, Paramyxoviridae infection includes infections by human metapneumovirus, respiratory syncytial virus, Mumps virus, Measles virus or parainfluenza viruses (e.g., type 1, 2, 3 and 4). In other embodiments, the virus of the Subfamily Pneumovirinae is a bovine respiratory syncytial virus or an avian pneumovirus.

As used herein, the term “protease activated receptor-I”, “proteinase activated receptor-1”, “PAR1” or “PAR-I” are used interchangeably. PAR1 is a G-protein-coupled receptor that is activated by thrombin cleavage thereby exposing an N-terminal tethered ligand. PAR1 is also known as “thrombin receptor” and “coagulation factor II receptor precursor” (see, for example, Vu, et al., Cell (1991) 64(6): 1057-68; Coughlin, et al, J Clin. Invest (1992) 89(2):351-55). The term PAR1 may include naturally occurring PAR1 and variants and modified forms thereof. The PAR1 can be from any source, but typically is a mammalian (e.g., human and non-human primate) PAR1, particularly a human PAR1. The nucleotide and amino acid sequences of PAR1 are known in the art. See, for example, Vu, et al., Cell (1991) 64(6): 1057-68; Coughlin, et al, J Clin Invest (1992) 89(2):351-55; and GenBank Accession number NM_{—}001992. The nucleic acid sequence of human PAR1 is available as GenBank™ accession number NM_{—}001992 {see also, M62424.1 and gi45O3636). The amino acid sequence of human PAR1 is available under accession number NP 001983 and AAA36743.

As used herein, the terms “treatment” or “treating” of a subject includes the application or administration of a suitable compound, or composition of the invention as defined herein to a subject (or application or administration of a compound or composition of the invention to a cell or tissue of a subject) with the purpose of delaying, stabilizing, curing, healing, alleviating, relieving, altering, remedying, ameliorating, improving, or affecting the disease or condition, the symptom of the disease or condition, the risk of (or susceptibility to) the disease or condition, or complication(s) of the disease or condition. The term “treating” refers to any indicia of success in the treatment or amelioration of an injury, pathology or condition, including any objective or subjective parameter such as abatement, remission, slowing disease progression or severity, stabilization, diminishing symptoms, or making the injury, pathology or condition more tolerable to the subject, slowing in the rate of degeneration or decline, making the final point of degeneration less debilitating, or improving a subject's physical or mental well-being. In some embodiments, the term “treating” includes increasing a subject's life expectancy, reducing morbidity and/or reducing mortality associated with a Paramyxoviridae infection. In some embodiments, the term “treating” includes reducing inflammation, preventing weight loss, increasing survival, inhibiting the progress of such a viral infection, reducing viral titers associated with Paramyxoviridae infections and/or reducing frequency or severity of associated complications such as viral or secondary bacterial pneumonia.

As used herein, “preventing” or “prevention” or “prophylactic treatment” is intended to refer to at least the reduction of likelihood of the risk of (or susceptibility to) acquiring a disease or disorder (i.e., causing at least one of the clinical symptoms of the disease not to develop in a subject that may be exposed to or predisposed to the disease but does not yet experience or display symptoms of the disease). Biological and physiological parameters for identifying such patients are provided herein and are also well known by physicians. In particular, “prevention” or “prophylactic treatment” of Paramyxoviridae infections may refer to the administration of the compounds of the present invention that prevent the symptoms of such infections. For prophylaxis purposes, the PAR1 inhibitor (e.g., antagonist) may be given preventively in those at risk during the winter-spring season or during an outbreak of Paramyxoviridae infection in the community.

In particular embodiments, the term “prevention or treatment of a Paramyxoviridae infection” includes: blocking or reducing the entry of Paramyxoviridae viruses into host cells (e.g. mammalian or avian); inhibiting the binding of to host cells; inhibiting replication of Paramyxoviridae viruses in infected host cells; reducing Paramyxoviridae viruses titers in the infected host, reducing inflammation. Accordingly, related aspects of the invention concerns the uses of PAR1 inhibitors for blocking entry, reducing entry, inhibiting the binding to, inhibiting replication and reducing titers of Paramyxoviridae viruses. In particular embodiments, the methods, compounds and composition of the invention are for addressing infections by Paramyxoviridae viruses, preferably viruses of the Subfamily Pneumovirinae, including but not limited to human respiratory syncytial virus (hRSV) and human metapneumovirus (hMPV).

### C) Compounds

In accordance with some embodiments, the compound for use in the methods and compositions of the invention is a Protease-Activated Receptor-1 (PAR1) inhibitor.

Advantageously, the compounds of the invention target the host instead of the virus, which may be useful in preventing emergence of virus resistance.

The term “inhibitor” as used herein, refers to a compound that is capable of inhibiting, directly or indirectly, the function or activity of PAR1, whether by binding or not to the PAR1 receptor. The term inhibitor encompasses the term antagonist and in preferred embodiment, PAR1 inhibitor is a PAR1 antagonist.

The term “antagonist” as used herein, refers to compound that is capable of specifically binding and inhibiting signaling through a receptor to fully block or detectably inhibit a response mediated by the receptor. For example, as used herein the term “PAR1 antagonist” is a natural or synthetic compound which binds and inactivates PAR1, fully or partially, thereby initiating or interfering with pathway signalling and further biological processes associated with PAR1 activity.

A PAR1 inhibitor (e.g., antagonist) according to the invention may be a peptide, a peptide mimetic, a small molecule organic compound (natural or chemically synthesized), an aptamer, a siRNA, a pepducin, a polynucleotide or an antibody.

As series of existing PAR1 antagonists are known, such as those described in Adams N. M. et al., Structure, function and pathophysiology of protease activated receptors, Pharmacology & Therapeutics (2011), doi:10.1016/jpharmthera.2011.01.003; and Lee H. and Hamilton J. R., Physiology, pharmacology, and therapeutic potential of protease-activated receptors in vascular disease, Pharmacology & Therapeutics (2012), doi:10.1016/j.pharmthera.2012.01.007, the content of which is incorporated herein by reference.

Existing and additional suitable PAR1 inhibitors or PAR1 antagonists according to the invention can be readily identified by those skilled in the art using various known methods. For instance, a PAR1 antagonist can be identified by its ability to bind to PAR1 and inhibit thrombin-induced calcium flux or thrombin-induced IL-8 production subsequent to intracellular signaling from a PAR1 (e.g., as measured in a FlipR assay, or by ELISA). Additional assays are described by Kawabata, et al., J Pharmacol Exp Ther. (1999) 288(1):358-70). In various embodiments a PAR1 antagonist of the invention provides at least about 10% less, or at least about 25% less, or at least about 50%, or at least about 75% less, or totally inhibits intracellular signalling from a control PAR1 not exposed to an antagonist, as measured by calcium flux or IL-8 production.

In embodiments, the PAR1 antagonist is a peptidomimetic, including, but not limited to the compound ([alpha]S)—W-[(1S)-3-amino-1[[{phenylmethyl)amino]carbonyl]propyl]-[alpha]-[[[[[1-(2,6-dichlorophenyl)methyl]-3-(1-pyrrolidinylmethyl)-1H-indol-6-yi]amino]carbonyl]amino]-3,4-difluorobenzenepropanamide, also known as “RWJ-56110”.

In other embodiments, the PAR1 inhibitor (e.g., antagonist) is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. In a particular embodiments, the PAR1 antagonist is selected from N3-cyclopropyl-7-{[4(I-methylethyl)phenyl]methyl)-7H-pyrrolo[3,2-f]quinazoline-I,3-diamine (SCH-79797, CAS 245520-69-8), Vorapaxar (SCH-530348; Shinohara et al. Journal of Stroke and Cerebrovascular Diseases (2012), Vol 21, No. 4, 318-324), Atopaxar (E5555; Goto et al. Eur Heart J (2010), 31, 2601-2613) and SCH-602539 (Chintala, M. et al Arterioscler Thromb Vasc Biol (2010), 30, 2143-2149).

### Antibodies

In another embodiment, the PAR1 inhibitor is an antagonist PAR1 antibody or an antigen binding fragment (antigen-binding molecule) (e.g. a blocking antibody). As used herein, unless otherwise defined, the term “antibody” includes both polyclonal and monoclonal antibodies, as well as antibody fragments having specific binding affinity for their antigen (antigen binding fragment), including, but not limited to, Fv fragments, Fab fragments, Fab′ fragments, F(ab)′2 fragments, and single chain (sFv) engineered antibody molecules. The term further includes, unless specifically excluded, chimeric and humanized antibodies, as well as human antibodies in circumstances where such antibodies can be produced.

Specific PAR1 antagonist antibodies have been disclosed in the art and these antibodies may be used according to the invention. See for instance R. R. Vassallo, Jr. et al. “Structure-Function Relationships in the Activation of Platelet Thrombin Receptors by Receptor-Derived Peptides,” J. Biol. Chem. 267:6081-6085 (1992) (“Vassallo, Jr. et al. (1992”)); L. F. Brass et al., “Structure and Function of the Human Platelet Thrombin Receptor,” J. Biol. Chem. 267: 13795-13798 (1992) (“Brass et al. (1992)”); and R. Kaufmann et al., “Investigation of PAR-1-Type Thrombin Receptors in Rat Glioma C6 Cells with a Novel Monoclonal Anti-PAR-I Antibody (Mab COR7-6H9), J. Neurocytol. 27:661-666 (1998) (“Kaufmann et al. (1998)”), which are incorporated herein by reference. Specific examples of potentially useful monoclonal antibodies according to the invention include, but are not limited to: the monoclonal antibody designated ATAP2 in Brass et al. (1992); the monoclonal antibody designated ATAP120 in Brass et al. (1992); and a monoclonal antibody designated ATAP138 in Brass et al. Additionally, monoclonal antibodies that may find a use in the compositions and methods according to the present invention include monoclonal antibodies that specifically bind either or both of the peptides used by Brass et al. (1992). Additionally, monoclonal antibodies that may be usable in compositions and methods according to the present invention include monoclonal antibodies that have complementary-determining regions that are identical to those of ATAP2, ATAP120, or ATAP138. Kaufmann et al. (1998) described monoclonal antibodies to rat PAR1 receptor that were prepared by using a peptide with a sequence described as being below the thrombin cleavage site for the receptor. Specific PAR1 antagonist antibodies according to the invention may include analogous antibodies can prepared against the corresponding region of human PAR1 receptor. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, N.Y., 1998; Kohler & Milstein, Nature 256:495-497 (1975); Kozbor et al., Immunology Today 4:72 (1983); and Cole et al., pp. 77-96 in Monoclonal Antibodies and Cancer Therapy, 1985.

In general, antibodies according to the present invention can be of any class, such as IgG, IgA, IgDI, IgE1, IgM1 or IgY1 although IgG antibodies are typically preferred. Antibodies can be of any mammalian or avian origin, including human, murine (mouse or rat), donkey, sheep, goat, rabbit, camel, horse, or chicken. In some alternatives, the antibodies can be bispecific. The antibodies can be modified by the covalent attachment of any type of molecule to the antibody. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, or other modifications known in the art. Monoclonal antibodies can be prepared using a wide variety of techniques known in the art including the use of hybridoma, recombinant, and phage display technologies, or a combination thereof.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. For example, suitable antibodies can be produced by phage display or other techniques.

Additionally, and not by way of limitation, human antibodies can be made by a variety of techniques, including phage display methods using antibody libraries derived from human immunoglobulin sequences and by the use of transgenic mice that are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. The antibodies can also be produced by expression of polynucleotides encoding these antibodies.

Additionally, antibodies according to the present invention can be fused to marker sequences, such as a peptide tag to facilitate purification; a suitable tag is a hexahistidine tag. The antibodies can also be conjugated to a diagnostic or therapeutic agent by methods known in the art. Techniques for preparing such conjugates are well known in the art. Other methods of preparing these monoclonal antibodies, as well as chimeric antibodies, humanized antibodies, and single-chain antibodies, are known in the art.

### Suppressors of PAR1 Expression

In addition to compounds which inhibit or suppress PAR1 biochemical or signaling activities, compounds which are capable of suppressing PAR1 expression or down-regulating PAR1 cellular levels may also be useful in the practice of the present invention. Suppression of PAR1 expression or down-regulation of its cellular level refers to a decrease in or an absence of PAR1 expression in an examined cell (e.g., a cell which has been contacted with a PAR1 antagonist compound), as compared to PAR1 in a control cell (a cell not treated with the PAR1 antagonist compound). PAR1 level or expression can be decreased or reduced by at least about 10% (e.g., by 20%, 30%, 40%, 50%, 60%, 70%, 80%, or 90%), as compared to PAR1 level or expression in the control cell. As indicated above, suppression of expression or down-regulation of PAR1 cellular levels can be carried out at either the level of transcription of the gene for PAR1 into mRNA or the translation of mRNA for PAR1 into the corresponding protein.

In some embodiments, inhibitory nucleotides are used to antagonize PAR1 mediated cardiac remodeling or other effects of PAR1 by suppressing PAR1 expression. These include short interfering RNA (siRNA), microRNA (miRNA), and synthetic hairpin RNA (shRNA), anti-sense nucleic acids, or complementary DNA (cDNA).

In some embodiments, a siRNA targeting PAR1 expression is used. Interference with the function and expression of endogenous genes by double-stranded RNA such as siRNA is known and has been shown in various organisms. siRNAs can include hairpin loops comprising self-complementary sequences or double stranded sequences. siRNAs typically have fewer than 100 base pairs and can be, e.g., about 30 bps or shorter, and can be made by approaches known in the art, including the use of complementary DNA strands or synthetic approaches. Such double-stranded RNA can be synthesized by in vitro transcription of single-stranded RNA read from both directions of a template and in vitro annealing of sense and antisense RNA strands. Double-stranded RNA targeting PAR1 can also be synthesized from a cDNA vector construct in which a PAR1 gene (e.g., human PAR1 gene) is cloned in opposing orientations separated by an inverted repeat. Following cell transfection, the RNA is transcribed and the complementary strands reanneal. Double-stranded RNA targeting the PAR1 gene can be introduced into a cell (e.g., a tumor cell) by transfection of an appropriate construct.

Typically, RNA interference mediated by siRNA, miRNA, or shRNA is mediated at the level of translation; in other words, these interfering RNA molecules prevent translation of the corresponding mRNA molecules and lead to their degradation. It is also possible that RNA interference may also operate at the level of transcription, blocking transcription of the regions of the genome corresponding to these interfering RNA molecules.

The structure and function of these interfering RNA molecules are well known in the art. In addition to double stranded RNAs, other nucleic acid agents targeting PAR1 can also be employed in the practice of the present invention, e.g., antisense nucleic acids. Since the PAR1 polynucleotide sequences from human and many other mammals have all been delineated in the art, inhibitory nucleotides (e.g., siRNA, miRNA, or shRNA) targeting PAR1 can be readily synthesized using methods well known in the art.

Exemplary siRNAs according to the invention could have up to 29 bases or bps, 25 bps, 22 bps, 21 bps, 20 bps, 15 bps, 10 bps, 5 bps or any integral number of base pairs between these numbers.

### Additional PAR1 Inhibitors

Other Exemplary PAR1 inhibitors (e.g., antagonists) that are contemplated by the invention include but are not limited to those described in:

U.S. Pat. No. 6,017,890 (Hoekstra et al.: “Azole Peptidomimetics as Thrombin Receptor Antagonists”), which is herein incorporated by reference in its entirety, and is specifically incorporated by reference for its teachings of compounds that function as thrombin receptor antagonists (see, e.g., column 2, line 31, through end of column 3 and Examples 1-10).

U.S. Pat. No. 5,446,131 (to Maraganore: “Thrombin Receptor Antagonists”), which is herein incorporated by reference in its entirety, and is specifically incorporated by reference for its teachings of compounds that function as thrombin receptor antagonists (see, e.g., the Abstract and the Claims).

U.S. Pat. No. 5,866,681 (to Scarborough: “Thrombin Receptor Antagonists”), which is herein incorporated by reference in its entirety, and is specifically incorporated by reference for its teachings of compounds that function as thrombin receptor antagonists (see, e.g., the Abstract, the Claims, and Examples 1-16).

U.S. Pat. No. 5,759,994 (to Coughlin: “Recombinant Thrombin Receptor and Related Pharmaceuticals”), which is herein incorporated by reference in its entirety, and is specifically incorporated by reference for its teachings of compounds that function as thrombin receptor antagonists (see, e.g., Examples 5 and 6, and the Claims).

U.S. Pat. No. 5,798,248 (to Coughlin: “Recombinant Thrombin Receptor and Related Pharmaceuticals”), which is herein incorporated by reference in its entirety, and is specifically incorporated by reference for its teachings of compounds that function as thrombin receptor antagonists (see, e.g., Examples 5 and 6, and the Claims).

Bematowicz et al. (“Development of Potent Thrombin Receptor Antagonists” J. Med. Chem. 39: 4879-4887, 1996), which is herein incorporated by reference in its entirety, and is specifically incorporated by reference for its teachings of compounds that function as thrombin receptor antagonists (see, e.g., Tables 1-8).

Vassallo et al. (“Structure-Function Relationships in the Activation of Platelet Thrombin Receptors by Receptor-Derived Peptides.” J. Biol. Chem. 267: 6081-6085, 1992), which is herein incorporated by reference in its entirety, and is specifically incorporated by reference for its teachings of compounds that function as thrombin receptor antagonists (see, e.g., Table 1).

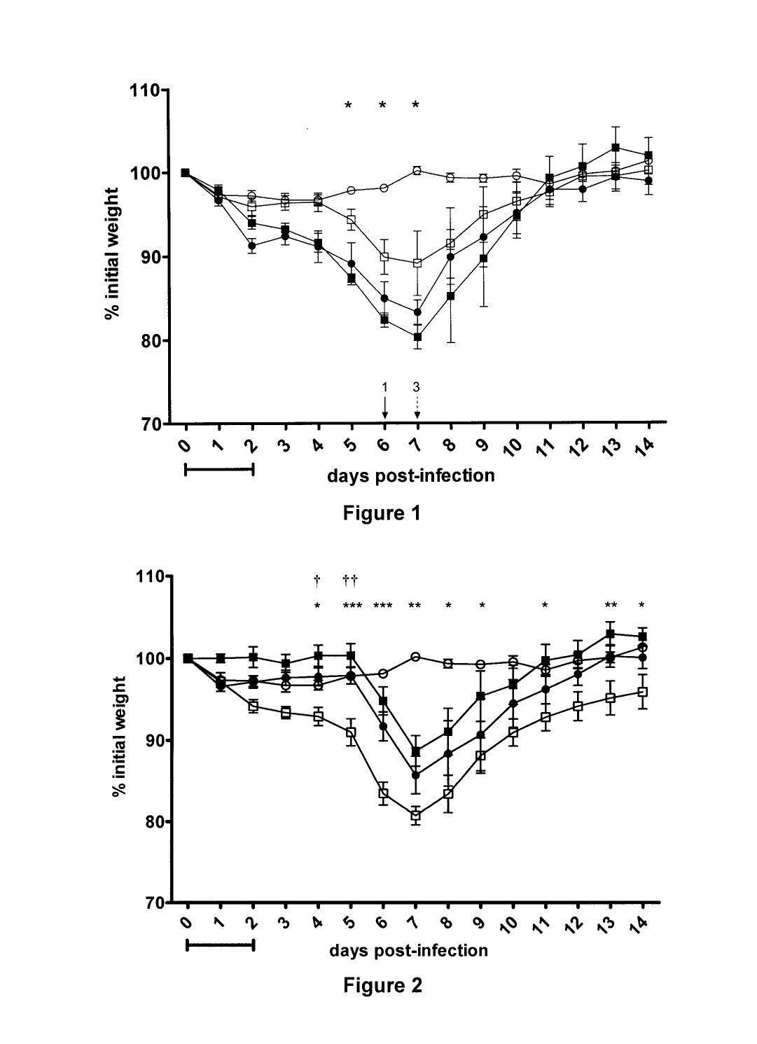

Andrade-Gordon et al. (“Design, Synthesis, and Biological Characterization of a Peptide-Mimetic Antagonist for a Tethered-Ligand Receptor.” Proc. Nat. Acad. Sci. USA 96: 12257-12262, 1999), which is herein incorporated by reference in its entirety, and is specifically incorporated by reference for its teachings of compounds that function as thrombin receptor antagonists (see, e.g., FIG. 1).

Hoekstra et al. (“Thrombin Receptor (PAR-1) Antagonists. Heterocycle-Based Peptidornirnetics of the SFLLR Agonist Motif.” Bioorg. Med. Chem. Lett. 8: 1649-1654, 1998), which is herein incorporated by reference in its entirety, and is specifically incorporated by reference for its teachings of compounds that function as thrombin receptor antagonists (see, e.g., Tables 1 and 2).

Kato et al. (“In Vitro Antiplatelet Profile of FR171113, a Novel Non-Peptide Thrombin Receptor Antagonist. “Euro. J. Pharmacol. 384: 197-202, 1999), which is herein incorporated by reference in its entirety, and is specifically incorporated by reference for its teachings of compounds that function as thrombin receptor antagonists (see, e.g., FIG. 1).

Ruda et al. (“Identification of Small Peptide Analogues Having Agonist and Antagonist Activity at the Platelet Thrombin Receptor.” Biochem. Pharmacol. 37:2417-2426, 1988), which is herein incorporated by reference in its entirety, and is specifically incorporated by reference for its teachings of compounds that function as thrombin receptor antagonists (see, e.g., the Abstract and FIG. 1).

Ruda et al. (“Thrombin Receptor Antagonists: Structure-Activity Relationships for the Platelet Thrombin Receptor and Effects on Prostacyclin Synthesis by Human Umbilical Vein Endothelial Cells.” Biochem. Pharmacol. 39:373-381, 1990), which is herein incorporated by reference in its entirety, and is specifically incorporated by reference for its teachings of compounds that function as thrombin receptor antagonists (see, e.g., Table 2).

Harmon and Jamieson (“Activation of Platelets by Alpha-Thrombin is a ReceptorMediated Event. J. Biol. Chem. 261: 15928-15933, 1986), which is herein incorporated by reference in its entirety, and is specifically incorporated by reference for its teachings of compounds that function as thrombin receptor antagonists (see, e.g., the abstract at page 15928, left column).

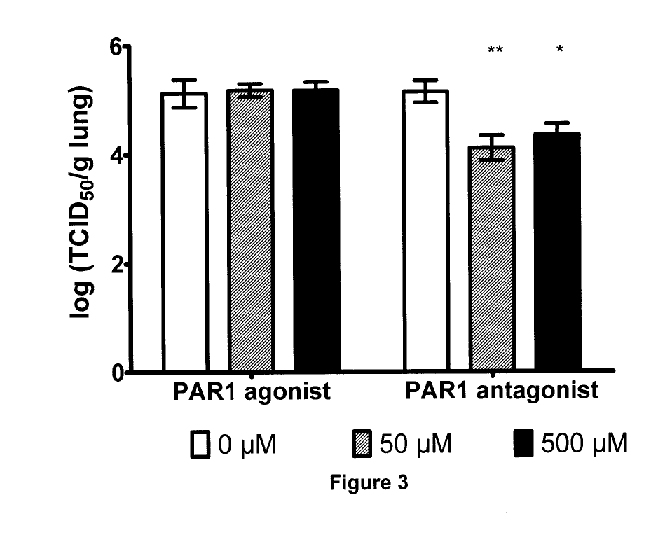

Doorbar and Winter (Isolation of a Peptide Antagonist to the Thrombin Receptor Using Phage Display. J. Mol. Biol. 244: 361-369, 1994), which is herein incorporated by reference in its entirety, and is specifically incorporated by reference for its teachings of compounds that function as thrombin receptor antagonists (see, e.g., FIG. 3).

Ahn et al. (Structure-Activity Relationships of Pyrroloquinazolines as Thrombin Receptor Antagonists. Bioorg. Med. Chem. Lett. 9: 2073-2078, 1999), which is herein incorporated by reference in its entirety, and is specifically incorporated by reference for its teachings of compounds that function as thrombin receptor antagonists (see, e.g., Tables 1 and 2).

Seiler et al. (Inhibition of Thrombin and SFLLR-Peptide Stimulation of Platelet Aggregation, Phospholipase A2 and Na+/H+ Exchange by a Thrombin Receptor Antagonist. Biochem. Pharmacol. 49: 519-528, 1995), which is herein incorporated by reference in its entirety, and is specifically incorporated by reference for its teachings of compounds that function as thrombin receptor antagonists (see, e.g., the Abstract).

Elliot et al. (Photoactivatable Peptides Based on BMS-197525: A Potent Antagonist of the Human Thrombin Receptor (PAR-1). Bioorg. Med. Chem. Lett. 9: 279-284, 1999), which is herein incorporated by reference in its entirety, and is specifically incorporated by reference for its teachings of compounds that function as thrombin receptor antagonists (see, e.g., Table 1).

Fujita et al. (A Novel Molecular Design of Thrombin Receptor Antagonists. Bioorg. Med. Chem. Lett. 9: 1351-1356, 1999), which is herein incorporated by reference in its entirety, and is specifically incorporated by reference for its teachings of compounds that function as thrombin receptor antagonists (see, e.g., the Abstract).

Debeir et al. (“Pharmacological Characterization of Protease-Activated Receptor (PAR-1) in Rat Astrocytes.” Euro. J. Pharmacol. 323: 111-117, 1997), which is herein incorporated by reference in its entirety, and is specifically incorporated by reference for its teachings of compounds that function as thrombin receptor antagonists (see, e.g., the Abstract).

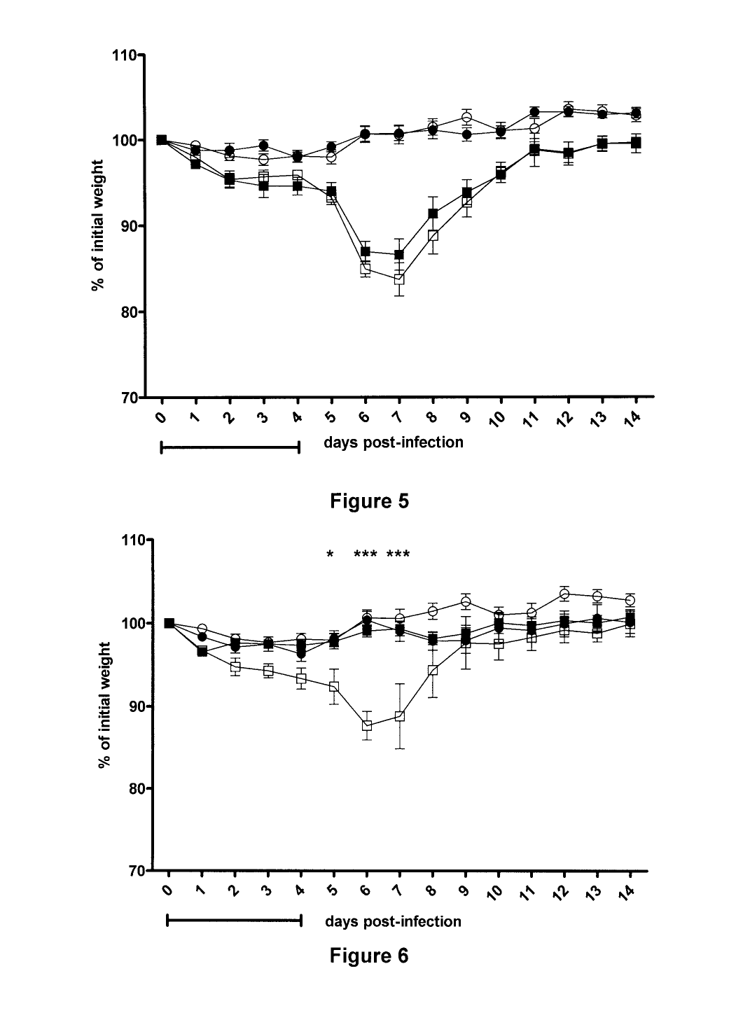

Ahn et al. (“Binding of a Thrombin Receptor Tethered Ligand Analogue to Human Platelet Thrombin Receptor.” Mol. Pharmacol. 51: 350-356, 1997), which is herein incorporated by reference in its entirety, and is specifically incorporated by reference for its teachings of compounds that function as thrombin receptor antagonists (see, e.g., FIG. 5 and Table 1).

McComsey et al. (“Reterocyc1e-peptide hybrid compounds. Aminotriazole-containing agonists of the thrombin receptor (PAR-1)”. Bioorganic & Medicinal Chemistry Letters 9: 1423-1428, 1999), which is herein incorporated by reference in its entirety, and is specifically incorporated by reference for its teachings of compounds that function as thrombin receptor antagonists (see, e.g., Table: Biological Data).

Nantermet et al. (“Discovery of a small molecule antagonist of the human platelet thrombin receptor (PAR-1).” Bioorganic & Medicinal Chemistry Letters 12: 319-323, 2002), which is herein incorporated by reference in its entirety, and is specifically incorporated by reference for its teachings of compounds that function as thrombin receptor antagonists (see, e.g., Table 1, Table 2, Table 3).

Barrow et al. (“Discovery and initial structure-activity relationship of trisubstituted ureas as thrombin receptor (PAR-1) antagonists.” Bioorganic & Medicinal Chemistry Letters 11: 2691-2696, 2001), which is herein incorporated by reference in its entirety, and is specifically incorporated by reference for its teachings of compounds that function as thrombin receptor antagonists (see, e.g., Table 1-5).

Ahn et al. (“Inhibition of cellular action of thrombin by N3-cyclopropyl-7 [[4-(1-methylethyl)phenyl]methyl]-7H-pyrrole[3,2f]quinazoline1,3-diamine (SCH79797), a non-peptide thrombin receptor antagonist.” Biochemical Pharmacol 60: 1425-1434, 2000), which is herein incorporated by reference in its entirety, and is specifically incorporated by reference for its teachings of compounds that function as thrombin receptor antagonists (see, e.g., FIG. 1).

Chackalamannil (“Thrombin receptor antagonists as novel therapeutic targets.” Curr Opin Drug Discovery Development 4: 417-427, 2001), which is herein incorporated by reference in its entirety, and is specifically incorporated by reference for its teachings of compounds that function as thrombin receptor antagonists.

Stead et al. (“Eryloside F, a novel penasterol disaccharide possessing potent thrombin receptor antagonist activity.” Bioorg. Med. Chem. Lett. 10: 661-664, 2000), which is herein incorporated by reference in its entirety, and is specifically incorporated by reference for its teachings of compounds that function as thrombin receptor antagonists (see, e.g., FIG. 1).

Pakala et al. (“A peptide analogue of thrombin receptor-activating peptide inhibits thrombin and thrombin-receptor-activating peptide induced vascular smooth muscle cell proliferation.” J. Cardiovasc. Pharmacol. 37: 619-629, 2001), which is herein incorporated by reference in its entirety, and is specifically incorporated by reference for its teachings of compounds that function as thrombin receptor antagonists (see, e.g., FIGS. 1 and 2).

Zhang et al. (“Discovery and optimization of a novel series of thrombin receptor (PAR-I) antagonists: potent, selective peptide mimetics based on indole and indazole templates.” J. Med. Chem. 44: 1021-1024, 2001), which is herein incorporated by reference in its entirety, and is specifically incorporated by reference for its teachings of compounds that function as thrombin receptor antagonists.

Adams N. M. et al., Structure, function and pathophysiology of protease activated receptors”, Pharmacology & Therapeutics (2011), doi:10.1016/jpharmthera.2011.01.003), which is incorporated herein by reference in its entirety, and is specifically incorporated by reference for its teachings of PAR1 antagonists in clinical trial such as: RWJ-56110, RWJ-58259 and SCH530348 (Chackalamannil et al., 2008 J Med Chem 51, 3061-3064; Clinical trial registration number: NCT00684203, NCT00684515, NCT00132912; Goto et al., 2010; J Atheroscler Thromb 17, 156-164; Macaulay et al., 2010 Expert Opin Pharmacother 11, 1015-1022; Thrombin receptor antagonist for clinical event reduction: TRA-CER Trial; Clinical trial registration number: NCT00527943; TRA*CER 2009; Clinical trial registration number: NCT00526474; Morrow et al., 2009 Am Heart J 158(335-341), all incorporated herein by reference) as well as another compound E5555 (Clinical trial registration numbers: NCT00619164 and NCT00548587; Clinical trial registration numbers: NCT00540670 and NCT00312052; Cirino & Severino, 2010 Expert Opin Thera Pat 2010 July; 20(7):875-84; Serebruany et al., 2009 Thromb Haemost 102, 111-119 all incorporated herein by reference).

### D) Pharmaceutical Compositions and Formulations

A related aspect of the invention concerns pharmaceutical compositions comprising one or more of the compounds of the invention described herein.

As used herein, the term “pharmaceutical composition” refers to the presence of at least one compound of the invention as defined herein and at least one pharmaceutically acceptable carrier or vehicle. The pharmaceutical compositions of the present invention are formulated by methods known to those skilled in the art. Suitable compositions may include solids, liquids, oils, emulsions, gels, aerosols, inhalants, capsules, pills, patches and suppositories. Compositions comprising compounds of the invention may be formulated as free base or pharmacologically acceptable salts. For instance, some PAR1 inhibitor (e.g., antagonist) may be formulated into a composition in a neutral or salt form. Pharmaceutically acceptable salts include the acid addition salts (formed with the free amine groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In a related aspect, the invention concerns pharmaceutical compositions comprising a compound as defined herein, and more particularly compositions formulated as an antiviral drugs. The invention further relates to the use of a compound as defined herein for the manufacture of a medicine for preventing and/or treating a Paramyxoviridae infection in a human subject, preferably a medicine for preventing and/or treating a Pneumovirinae infection.

“Pharmaceutically acceptable vehicle” refers to a diluent, adjuvant, excipient, or carrier with which a compound is administered. The term “pharmaceutically acceptable” refers to drugs, medicines, inert ingredients etc., which are suitable for use in contact with the tissues of humans and lower animals without undue toxicity, incompatibility, instability, irritation, allergic response, and the like, commensurate with a reasonable benefit/risk ratio. It preferably refers to a compound or composition that is approved or approvable by a regulatory agency of the Federal or State government or listed in the U.S. Pharmacopoeia or other generally recognized pharmacopoeia for use in animals and more particularly in humans. The pharmaceutically acceptable vehicle can be a solvent or dispersion medium containing, for example, water, ethanol, polyol (for example, glycerol, propylene glycol, and liquid polyethylene glycol), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms can be achieved by addition of antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

With respect to pharmaceutically useful compounds or compositions according to the present invention that is to be given to an individual, administration is preferably a “prophylactically effective amount” or a “therapeutically effective amount”.

In preferred embodiments, administering one or more of the compounds of the invention to a subject comprises administering a therapeutically effective amount. As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc), and the particular disorder(s) the subject is suffering from. In addition, the therapeutically effective amount may depend on the subject's blood parameters (e.g. lipid profile, insulin levels, glycemia), the severity of the disease state, organ function, or underlying disease or complications. Such appropriate doses may be determined using any available assays including the assays described herein. When one or more of the compounds of the invention is to be administered to humans, a physician may for example, prescribe a relatively low dose at first, subsequently increasing the dose until an appropriate response is obtained.

The dosage and frequency of administration can also vary depending on whether the treatment is prophylactic or therapeutic. In prophylactic applications, a relatively low dosage may be administered at relatively infrequent intervals over a long period of time. Some subjects may continue to receive treatment for the rest of their lives. In therapeutic applications, a relatively high dosage at relatively short intervals may sometimes be required until progression of the disease is reduced or terminated, and preferably until the subject shows partial or complete amelioration of symptoms of disease. Thereafter, the subject can be administered a prophylactic regime. In particular embodiments, the Protease-Activated Receptor-1 (PAR1) inhibitor is administered for at least one day, or for at least two days, or for at least three days, or for at least five days or for at least ten days or longer prior infection.

A sufficient amount of a PAR1 inhibitor (e.g., antagonist) may be any sufficient amount to treat or prevent Paramyxoviridae infections at a reasonable benefit/risk ratio applicable to any medical treatment. However, the daily dosage of the compound may be varied over a wide range from 0.01 to 1,000 mg per adult per day. In embodiments, the compositions contain 0.01, 0.05, 0.1, 0.5, 1.0, 2.5, 5.0, 10.0, 15.0, 25.0, 50.0, 100, 250 and 500 mg of the active ingredient (e.g. PAR1 inhibitor) for the symptomatic adjustment of the dosage to the patient to be treated. A medicine typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to particular prophylactic embodiments, compositions containing PAR1 inhibitor (e.g., antagonist) are administered to a subject (e.g. human patient) not already suffering from a Paramyxoviridae infection. Rather, the PAR1 inhibitor is administered to a subject who is at the risk of, or has a predisposition, to developing such an infection or associated disorder. Such preventive administration may permit to enhance the subject's resistance, block the infection or at least to retard the progression of the infection. In particular embodiments, the Protease-Activated Receptor-1 (PAR1) inhibitor is administered before infection or at any time after infection (preferably shortly) or shortly after appearance of symptoms of infection. For instance, in particular embodiments, the PAR1 inhibitor is administered at least 12 hours, or at least one day, or at least two days, or at least three days, or at least five days or at least ten days before infection. In particular embodiments, the PAR1 inhibitor is administered within one to 12 hours, within one day, within two days, within three days, within five days after likelihood of an infection or appearance of symptoms of such viral infection. The PAR1 inhibitor may be administered for at least one day, or for at least two days, or for at least three days, or for at least five days or for at least ten days or longer following infection.

The invention also encompasses the uses of a compound of the invention as defined herein, in combination with one or more existing antiviral drug. The pharmaceutical compositions of the invention may comprise a compound of the invention as defined herein, in combination with one or more existing antiviral drug. Examples of existing antiviral drugs include, but are not limited to: neuraminidase inhibitors such as Oseltamivir (Tamiflu™), Zanamivir (Relenza™), Laninamivir (Inavir™), and Peramivir. Accordingly, the invention encompasses antiviral compositions comprising a Protease-Activated Receptor-1 (PAR1) inhibitor in combination with a neuraminidase inhibitor. A compound of the invention may also be used in combination with one or more additional pharmaceuticals including, but not limited to, ribavirin, peginterferon alfa-2b, peginterferon alfa-2a, antibiotics, and anti-inflammatory compounds such as corticosteroids.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Intranasal administration may be preferred because that mode of administration generally has fewer side effects.

### E) Screening Assays

A further object of the invention relates a method for screening PAR1 antagonists for use in the treatment or prevention of Paramyxoviridae infections. For example, the screening method may measure the binding of a candidate compound to PAR1, or to cells or membranes bearing PAR1, or a fusion protein thereof by means of a label directly or indirectly associated with the candidate compound. Furthermore, the screening method may involve measuring, qualitatively detecting, or quantitatively detecting ability of said candidate compound to inactivate PAR1 and/or to interfere with the virus infection.

In a particular embodiment, the screening method of the invention comprises the step consisting of:
a) providing a plurality of cells expressing PAR1 on their surface (e.g. epithelial cells);
b) incubating said cells with a candidate compound;
c) determining whether said candidate compound binds to PAR1, inactivates PAR1 and/or interfere with virus infection;
d) selecting the candidate compound that binds to PAR1, inactivates PAR1 and/or interfere with virus infection.

The candidate compounds may be selected from a library of compounds previously synthesized, or a library of compounds for which the structure is determined in a database, or from a library of compounds that have been synthesized de novo or natural compounds. The candidate compound may be selected from the group of (a) proteins or peptides, (b) nucleic acids and (c) organic or chemical compounds (natural or not) including small organic molecules.

PAR1 inactivation with the candidate compound can be tested by various known methods of the man skilled in the art. In a particular embodiment, the screening method of the invention may further comprises a step of testing the candidate compound for its ability to treat or prevent Paramyxoviridae infections, for example by administering the candidate compound selected at step d) to an animal model of Paramyxoviridae infection to validate the protective effects of the candidate compound.

In general, such screening methods involve providing appropriate cells which express PAR1 on their surface. If necessary, the cells may be transfected to express PAR1 using methods well known in the art.

### F) Diagnostic Applications

A further object of the invention relates to a method of testing whether a subject is predisposed to a Paramyxoviridae infection, which comprises the step of analyzing a biological sample from the subject for: (i) detecting the presence of a mutation in the PAR1 gene and/or its associated promoter, and/or (ii) assessing the expression of the PAR1 gene.

Detecting the presence of a mutation in the PAR1 gene and/or its associated promoter comprises obtaining a biological sample (e.g. blood, serum, saliva, urine, etc) from the subject. Typical techniques for detecting a mutation in the PAR1 gene and/or assessing PAR1 gene expression may include the use of restriction fragment length polymorphism, hybridization techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridization, ligation chain reaction, mini-sequencing, DNA chips, allele-specific oligonucleotide hybridization with single or dual-labelled probes merged with PCR or with molecular beacons, and others.

In other embodiments, the expression of the PAR1 gene is assessed by analyzing the expression of the protein translated from the gene. Such analysis can be done by using a variety of techniques well known from one of skill in the art including, but not limited to, enzyme immunoassay (EIA), radioimmunoassay (RIA), Western blot analysis and enzyme linked immunoabsorbant assay (ELISA). Analyzing the expression of the protein translated from the gene may also comprises using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the PAR1 gene.

The method of the invention may comprise comparing the level of expression of the PAR1 gene in a biological sample from a subject with the normal expression level of said gene in a control. For instance, a significantly higher level of expression of the PAR1 gene in the biological sample of a subject as compared to the normal expression level may be an indication that the subject (e.g. human patient) is predisposed or more sensitive to developing a severe Paramyxoviridae infection.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further limiting.

### EXAMPLES

### Example 1

### PAR Experiments in Human Metapneumovirus (hMPV) and Human Respiratory Syncytial Virus (hRSV) Experimental Models

### Materials and Methods

### Virus Strains and Cells:

LLC-MK2 and Hep2 cells were maintained in minimal essential medium (MEM; Life Technologies) supplemented with 10% fetal bovine serum (FBS; Wisent). The hMPV A strain C-85473, a clinical strain that was passed nine times on LLC-MK2, was grown on LLC-MK2 cells in OptiMEM™ (Life technologies) supplemented with 0.0002% trypsin (Sigma). High virus titres were obtained by infecting 16 flasks (75 cm²) of LLC-MK2 cells until complete cytopathic effects were observed. Infected monolayers and supernatants were recovered with a cell scraper, sonicated and concentrated on Amicon™ columns (Fisher). The pooled preparation was centrifuged (1200 r.p.m., 10 min) to remove cellular debris. Supernatant was aliquoted and stored at −80° C. The same protocol was used with 16 flasks of uninfected cells for control mice.

The clinical hRSV A strain 15 959 that was passed nine times on Hep2 cells, was grown on Hep2 cells in MEM™ (Life Technologies) supplemented with 2% FBS (Wisent). Hep2 cells were infected with 10 TCID₅₀/Hep2 cell of hRSV 15 959 and plated out into one flask (150 cm²) until 60% cytopathic effects were observed. Infected monolayers and supernatants were recovered with a cell scraper and sonicated. The pooled preparation was centrifuged (1200 r.p.m., 10 min) to remove cellular debris. Supernatant was aliquoted and stored at −80° C. The same protocol was followed for a flask of uninfected cells for control mice.

### Viral Titers:

hMPV viral titers were determined by 10-fold serial dilutions of virus in 24-well plates containing LLC-MK2 cells. Before infection, cells were washed twice with phosphate-buffered saline (PBS) to remove residual serum proteins that could inhibit trypsin activity. Infected plates were incubated at 37° C. with 5% CO₂ and replenished with 1 μl of fresh trypsin (0.0002%) every other day. hRSV viral titers were determined by 10-fold serial dilutions of virus in 24-well plates containing Hep2 cells. Before infection, cells were washed twice with phosphate-buffered saline (PBS). Infected plates were incubated at 37° C. with 5% CO₂. On day 4 post infection, plates were fixed with 80% acetone at −20° C. for 30 min, washed with PBS and air dried. The presence of hRSV was detected by an immuno-staining assay using a monoclonal goat Anti-Respiratory Syncytial Virus primary antibody and a donkey anti-Goat IgG Secondary Antibody, HRP conjugate (both from Cederlane). Virus titers were reported as 50% tissue culture infectious doses (TCID₅₀) per ml, per mouse or per gram of lung. The lower limit of detection of the assay is 10² TCID₅₀ per gram. TCID₅₀ were calculated by the Reed and Muench method.

Compounds:

PAR-1 agonist (TFLLR-NH2 (SEQ ID NO.:1)) (Genescript) was reconstituted in H₂O at a concentration of 10 mM aliqoted and stored at −20° C. Immediately before intranasal administration, PAR-1 agonist was diluted to 50 μM or 500 μM in OptiMEM™. As a control, H₂O was diluted 1/20 in OptiMEM immediately before intranasal administration.

PAR-1 antagonist (SCH-79797: N3-cyclopropyl-7-{[4(I-methylethyl)phenyl]methyl)-7H-pyrrolo[3,2-f]quinazoline-I,3-diamine) (Axon MedChem) was reconstituted at 22 mM in DMSO and stored at −20° C. Immediately before intranasal administration, PAR-1 antagonist was diluted to 50 μM or 500 μM in OptiMEM™. As a control, DMSO was diluted 1/44 in OptiMEM™ immediately before intranasal administration.

Control peptide (FTLLR-NH2 (SEQ ID NO.:2)) (Genescript) was reconstituted in H₂O at a concentration of 10 mM aliqoted and stored at −20° C. Immediately before intranasal administration, the control peptide was diluted to 50 μM or 500 μM in OptiMEM. As a control, H₂O was diluted 1/20 in OptiMEM immediately before intranasal administration.

### Balb/C Mouse Studies:

In the first protocol, groups of 12 4-6-week-old female BALB/c mice (Charles River Laboratories) were infected intranasally with 4-7×10⁵ TCID₅₀ hMPV strain C-85473 in 25 μl of OptiMEM™ supplemented with the PAR-1 or PAR-1 antagonist at a final concentration of 50 μM or 500 μM. As infected controls, groups of 12 4-6-week-old BALB/c mice (Charles River Laboratories) were infected intranasally with 4-7×10⁵ TCID₅₀ hMPV strain C-85473 in 25 μl of OptiMEM™ supplemented with H₂O or DMSO. As an uninfected control, 12 mice were sham infected with the concentrated supernatant of non-infected LLC-MK2 cells supplemented with DMSO. On day 1 and 2 post infection, animals were treated with the PAR-1 agonist or PAR-1 antagonist at a final concentration of 50 μM or 500 μM, or H₂O or DMSO for control groups, resulting in a 3-day treatment in total. All animals were housed in groups of four in micro-isolator cages. The animals were evaluated on a daily basis for mortality, weight loss, and the presence of symptoms. On day 5 post-infection, lungs were removed from six mice per group for the evaluation of viral titres by cell culture and cytokine expression by Luminex™.

In the second protocol, groups of 18 4-6-week-old BALB/c mice (Charles River Laboratories) were infected intranasally with 6-8×10⁵ TCID₅₀ hMPV strain C-85473 in 25 μl of OptiMEM™ supplemented with the PAR-1 agonist, PAR-1 antagonist or control peptide at a final concentration of 500 μM. As infected controls, groups of 18 4-6-week-old BALB/c mice (Charles River Laboratories) were infected intranasally with 6-8×10⁵ TCID₅₀ hMPV strain C-85473 in 25 μl of OptiMEM™ supplemented with H₂O or DMSO. As uninfected control, groups 18 mice were sham infected with the concentrated supernatant of non-infected LLC-MK2 cells supplemented with the PAR-1 agonist, PAR-1 antagonist or control peptide at a final concentration of 500 μM or DMSO. On day 1 through 4 post infection, animals were treated with the PAR-1 agonist, PAR-1 antagonist or control peptide at a final concentration of 50 μM or 500 μM, or H₂O or DMSO for control groups, resulting in a 5-day treatment in total. Animals were housed in groups of five and three in micro-isolator cages. The animals were evaluated on a daily basis for mortality, weight loss, and the presence of symptoms. On day 5 post-infection, lungs were removed from six mice per group for the evaluation of viral titres by cell culture and cytokine expression by Iuminex™. Lungs of another six mice per group were removed for histopathological analysis.

In a third protocol, Two groups of 12 4-6-week-old BALB/c mice (Charles River Laboratories) were infected intranasally with 6×105 TCID50 hMPV strain C-85473 in 25 μl of OptiMEM™, supplemented with the PAR-1 agonist or PAR-1 antagonist at a final concentration of 500 μM and treated on days 1 through 4 post infection with the PAR-1 agonist or PAR-1 antagonist (500 μM). Two more groups of 4-6-week-old BALB/c mice (Charles River Laboratories) were infected intranasally with 6×105 TCID50 hMPV strain C-85473 in 25 μl of OptiMEM™. These groups were treated from day 1 though 5 post infection (treatment delayed 24 h post-infection) with the PAR-1 agonist or PAR-1 antagonist (500 μM). As infected controls, groups of 12 4-6-week-old BALB/c mice (Charles River Laboratories) were infected intranasally with 4×105 TCID50 hMPV strain C-85473 in 25 μl of OptiMEM supplemented with DMSO. All animals were housed in groups of four in micro-isolator cages. The animals were evaluated on a daily basis for mortality, weight loss, and the presence of symptoms. On day 5 post-infection, lungs were removed from six mice per group for the evaluation of viral titres by cell culture

Finally in a fourth protocol, groups of 12 4-6-week-old BALB/c mice (Charles River Laboratories) were infected intranasally with 2×105 TCID50 hRSV strain 15 595 in 25 μl of OptiMEM™ supplemented with the PAR-1 agonist or PAR-1 antagonist at a final concentration of 500 μM. On day 1 through 4 post infection, animals were treated with the PAR-1 agonist or PAR-1 antagonist at a final concentration of 500 μM, resulting in a 5-day treatment in total. As infected controls, groups of 12 4-6-week-old BALB/c mice (Charles River Laboratories) were infected intranasally with 2×105 TCID50 hRSV strain 15 595 in 25 μl of OptiMEM™ supplemented with DMSO. As uninfected control, groups 12 mice were sham infected with the concentrated supernatant of non-infected Hep2 cells. Animals were housed in groups of four in micro-isolator cages. The animals were evaluated on a daily basis for mortality, weight loss, and the presence of symptoms. On day 5 post-infection, lungs were removed from six mice per group for the evaluation of viral titres by cell culture.

### Pulmonary Viral Titers:

On day 5 post infection, six animals per group were sacrificed, the lungs were removed and snap frozen in liquid nitrogen. Lungs were weighed, homogenized in 1 ml of PBS, centrifuged (2000 rpm, 10 min) and the supernatant was used to infect LLC-MK2 monolayers (in case of hMPV-infected mice) or Hep2 monolayer (in case of hRSV-infected mice) for virus titration as reported in the section “Viral titers”.

### Pulmonary Cytokine Expressing:

250 μL of lung homogenates (see section “Pulmonary viral titers”) were added to 250 μl 50 mM KPO₄, pH 6.0 buffer containing 0.2% CHAPS {3-[(3-cholamidopropyl)-dimethylammonio]1-1-propanesulfonate} (Sigma) and 0.2% of a protease inhibitor cocktail (Sigma) and stored at −20° C. until the day of analysis. Samples were centrifuged at 13,800×g for 10 min at 4° C., and 50 μl of the supernatant was used for cytokine quantification. Levels of interleukin-4, IL-6, IL-12(p40), IL-12(p70), IFN-γ, KC, MCP-1, MIP-1α, RANTES were determined by the use of a 9-plex mouse bead kits (BioRad™/Millipore™) according to the manufacturer's instructions. Experiments were performed in a 96-well filter plate and results were analysed with the Luminex™ system (Qiagen™).

### Histopathology:

On day 5 post infection, 6 animals per group were sacrificed, and the lungs were collected at specified time points and fixed with 4% buffered formalin. Fixed lungs were subsequently embedded in paraffin, sectioned in slices of 5 μm, and stained with hematoxylin-eosin. The histopathological score was determined by an independent researcher who was blinded to experimental data. A semiquantitative scale was used to score bronchial/endobronchial, peribronchial, perivascular, interstitial, pleural and intra-alveolar inflammation.

### Statistical Analysis:

Differences in weight loss between groups was analysed by repeated measures two-way ANOVA. Viral titers, cytokine levels and mean histopathology scores were analysed using student t-test.

### Results

In the three-day prophylactic treatment regimen (experiment 1), a dose-dependent increase in weight loss was observed for hMPV-infected mice treated with the PAR-1 agonist compared to hMPV infected, untreated mice that were given H₂O (1/20 diluted in OptiMEM™). This difference was significant on days 5 through 7 for mice treated with 500 μM of PAR-1 agonist. One out of 6 mice and 3 out of 6 mice treated with 50 μM and 500 μM of PAR-1 agonist, respectively, reached the humane endpoint compared to none in the untreated group (FIG. 1).

Conversely, a dose dependent reduction in weight loss was observed for hMPV infected mice treated with the PAR-1 antagonist compared to hMPV infected, untreated mice that were given DMSO (1/44 diluted in OptiMEM™) (FIG. 2).

A significant (though not dose-dependent) reduction in pulmonary viral titers was detected on day 5 post hMPV infection for mice treated with the PAR-1 antagonist compared to mice treated with diluted DMSO. No significant difference in pulmonary viral titers was observed for PAR-1 agonist treated, hMPV-infected mice compared to diluted H₂O treated mice (FIG. 3).

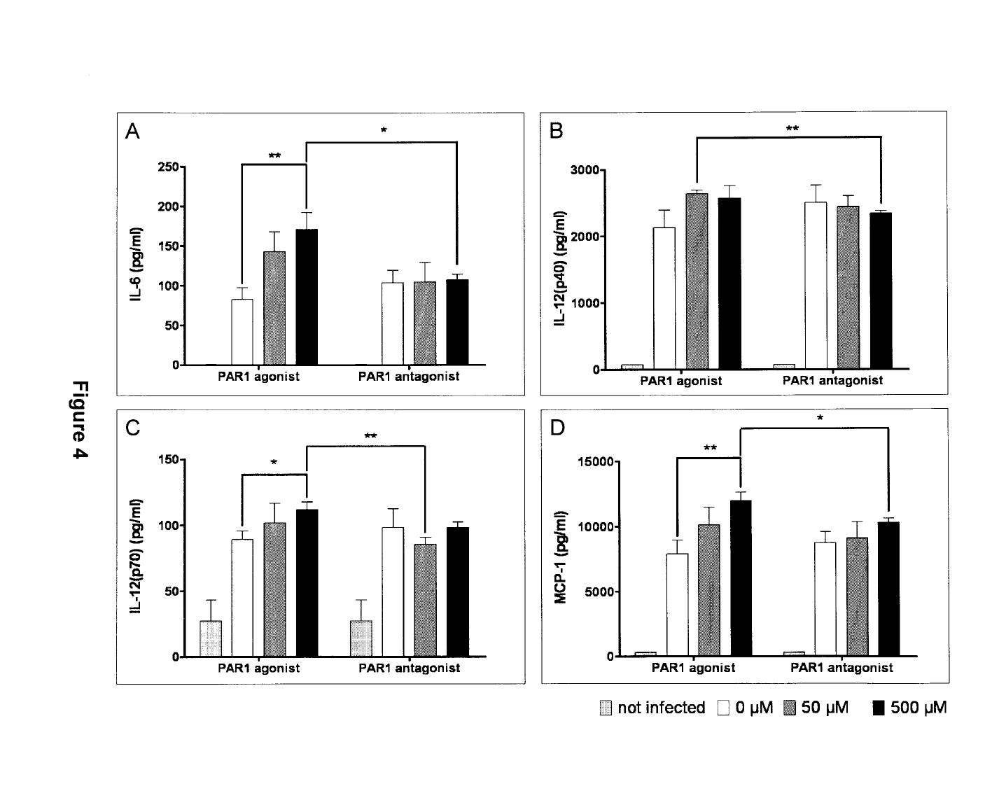

In the lungs of hMPV-infected, PAR-1 antagonist treated mice, no significant difference in cytokine expression was observed on day 5 post infection; however dose-dependent increases in IL-6, IL-12(p40) and MCP-1 were found in PAR-1 agonist treated mice (FIG. 4).

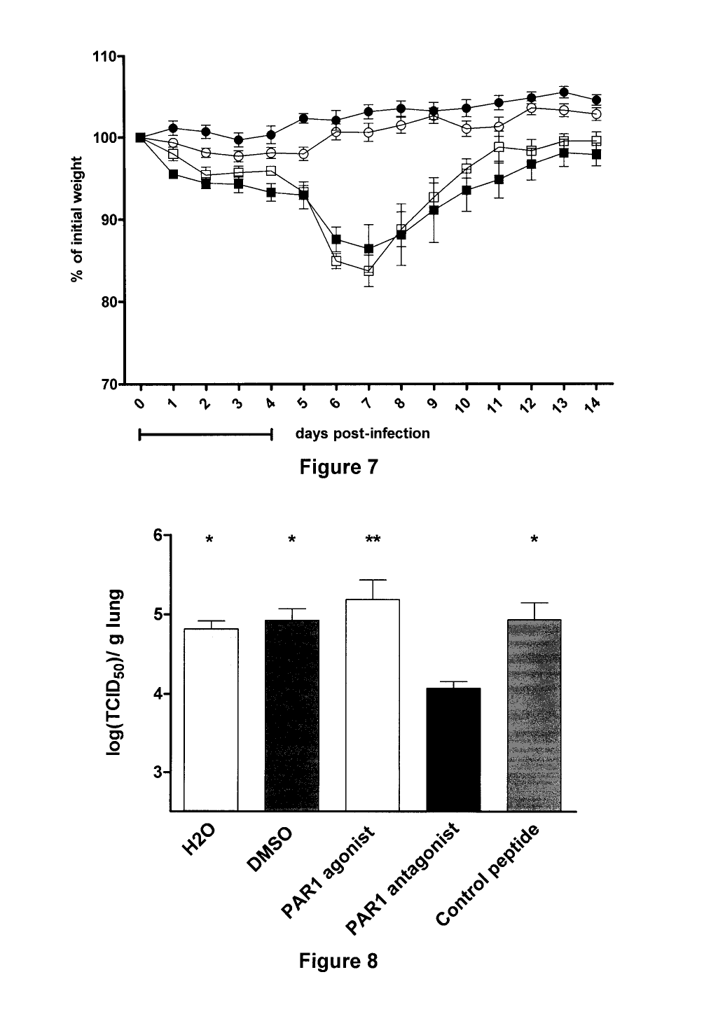

In the five-day pre-exposure prophylactic treatment regimen (experiment 2), no significant difference in weight loss was observed between hMPV-infected, PAR-1 agonist (500 μM) treated mice and hMPV-infected, H₂O-treated mice or hMPV infected mice treated with a control peptide (500 μM) (FIG. 5, FIG. 7). Importantly, an increase from 3 to 5 days of treatment abolished all weight loss and symptoms in hMPV-infected PAR-1 antagonist (500 μM) treated mice (FIG. 6). As a control, uninfected mice were treated for 5 days with either PAR-1 agonist, PAR-1 antagonist or the control peptide at a concentration of 500 μM. In none of the uninfected groups significant weight loss or symptoms were observed (FIG. 5, FIG. 6, FIG. 7).

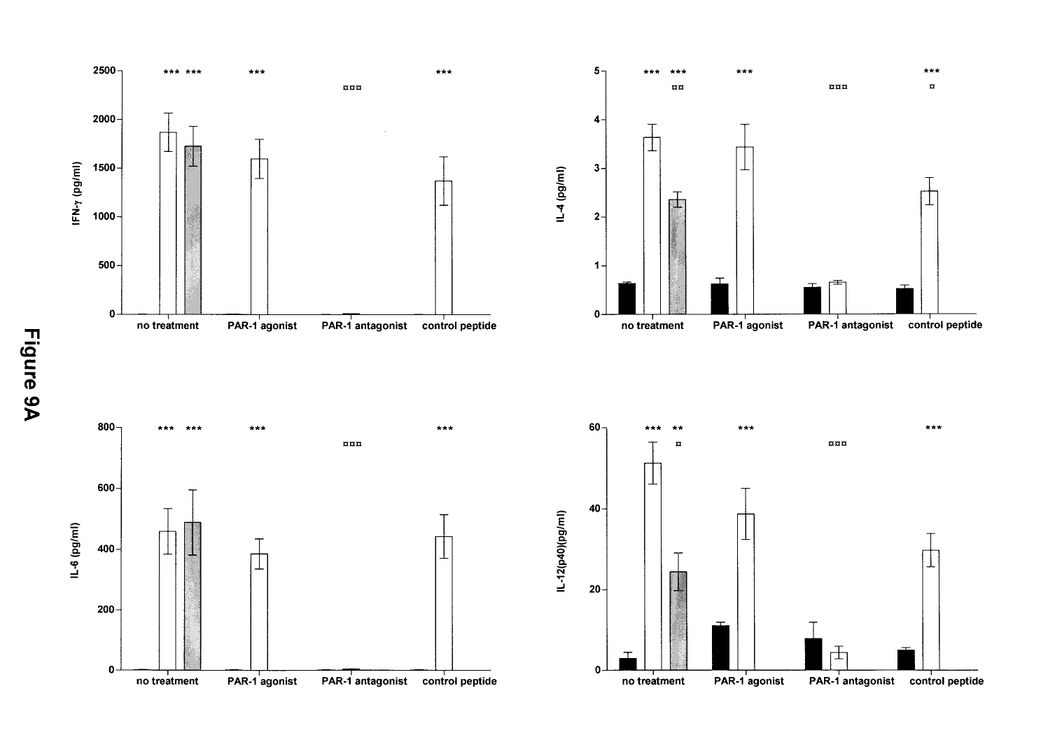

Compared to all other infected groups, reduced pulmonary viral titers were only observed for the hMPV-infected mice treated with the PAR-1 antagonist (FIG. 8).

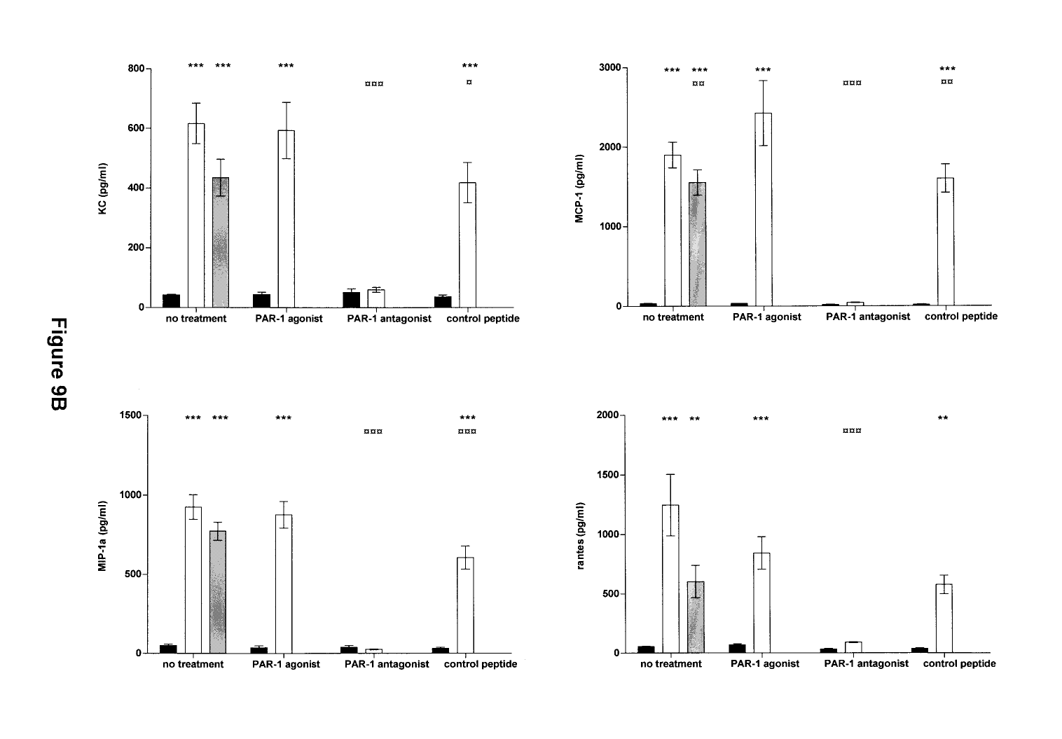

The pulmonary expression of 9 inflammatory cytokines/chemokines (IFN-γ, IL-4, IL-6, IL-12(p40), IL-12(p70), KC, MCP-1, MIP-1α and RANTES) was evaluated using a multiplex Luminex™ assay. No significant levels of IL-12(p70) were observed for any of the groups analysed. None of the uninfected groups showed significant changes in cytokine/chemokine levels. Infected mice treated with the PAR-1 agonist showed increased levels of IL-4, IL-12(p40) and MCP-1 compared to the infected mice treated with H₂O and increased levels of IL-4, KC, MCP-1 and MIP-1α compared to mice treated with the control peptide. A significant reduction in all of the evaluated inflammatory cytokine/chemokine levels was observed for infected mice treated with the PAR-1 antagonist, compared to all other groups of infected mice (FIG. 9).

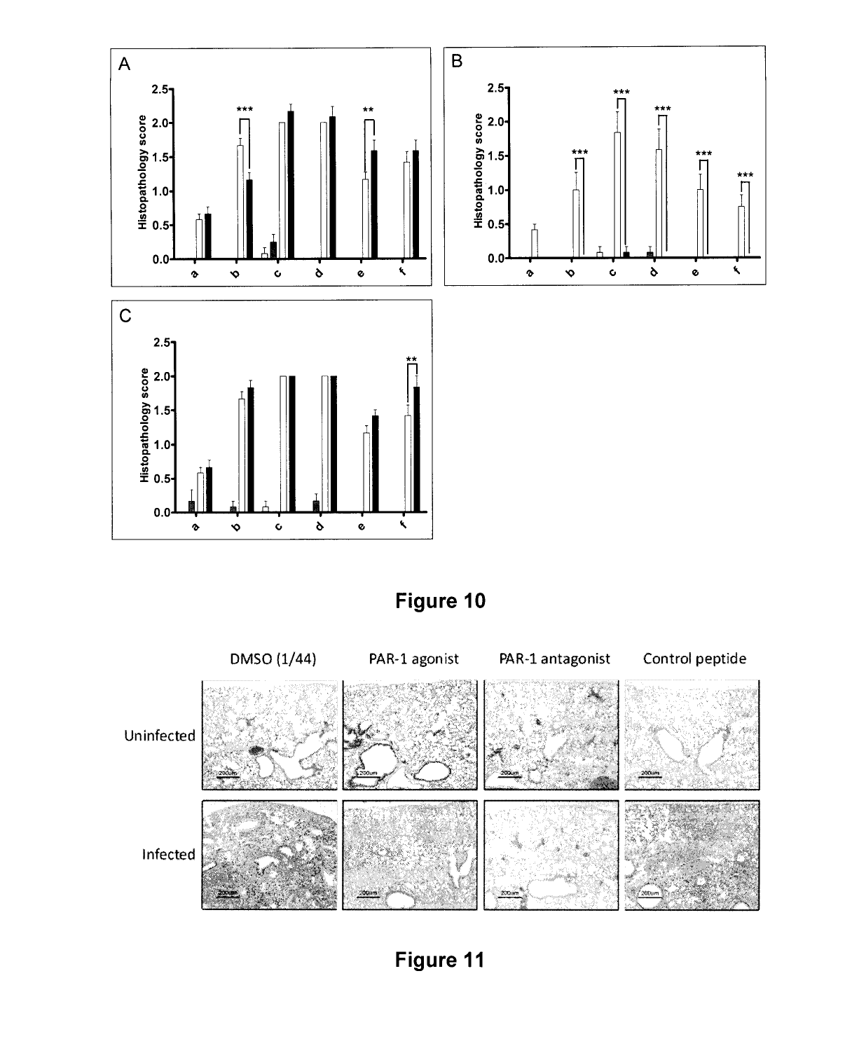

Histopathological analysis of lungs removed on day 5 post hMPV infection revealed increases in all but one (peribronchial) inflammation criteria for infected mice treated with the PAR-1 agonist compared to uninfected, untreated mice. In contrast no pulmonary inflammation was observed in infected mice, treated with the PAR-1 antagonist. Pulmonary inflammation was similar for infected untreated and infected, control peptide treated mice (FIG. 10, FIG. 11).

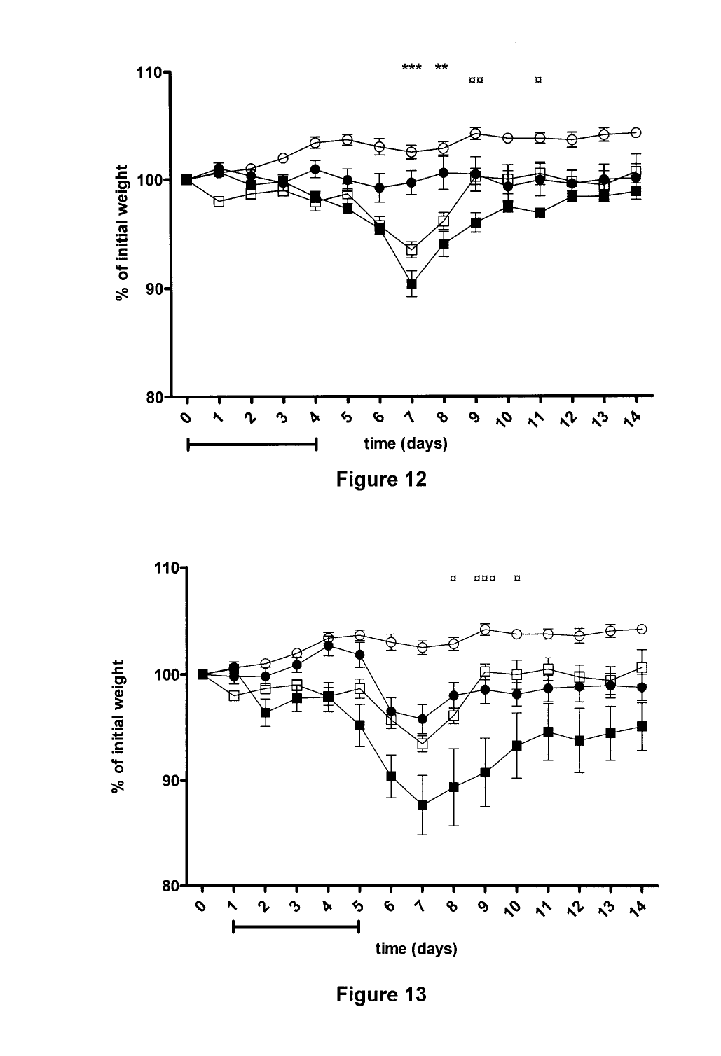

FIG. 12 shows the significant reduced weight loss achieved with a 5-day prophylactic treatment with the PAR-1 antagonist (500 μM) compared to the infected/untreated group. In contrast, Par-1 agonist (500 μM)-treated mice regained weight less rapidly than the control. In the five-day post-exposure treatment regimen (experiment 3), hMPV-infected mice, for which the 5-day treatment with the PAR-1 antagonist (500 μM) started 24 h post infection lost less weight than infected/untreated mice and weight loss started at a later time point. hMPV-infected mice, for which the 5-day treatment with the PAR-1 agonist (500 μM) started 24 h post infection regained their weight more slowly than infected/untreated mice (FIG. 13).

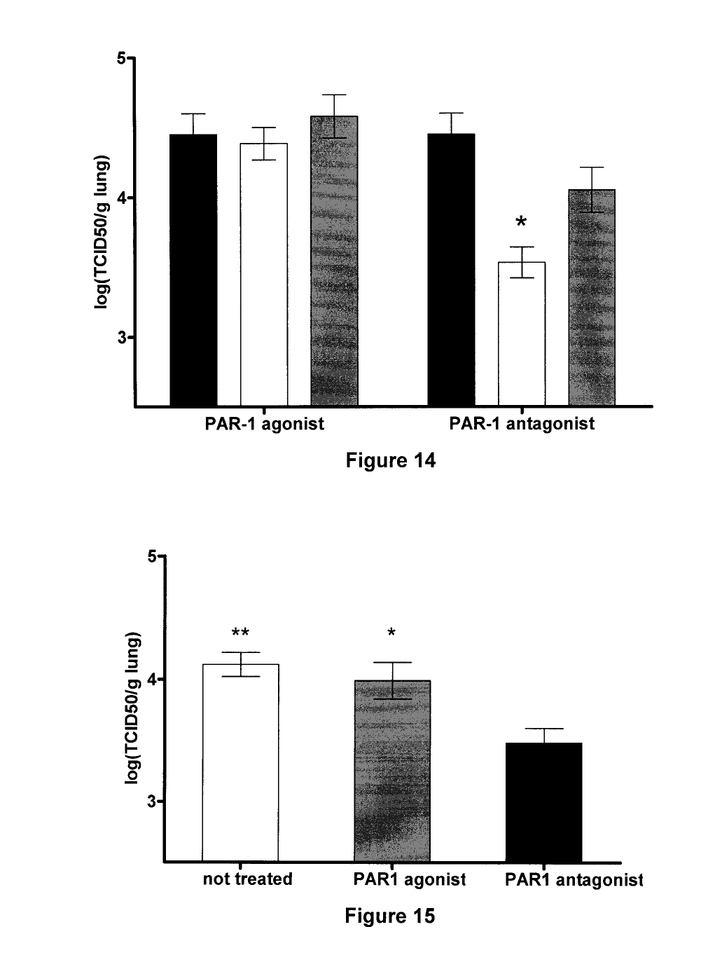

No significant difference in pulmonary viral titers was observed between mice for which treatment with the PAR-1 agonist was started either simultaneously or 24 h post infection. A reduction in pulmonary viral titers was observed in mice that received treatment with the PAR-1 antagonist and such reduction was statistically significant when the compound was given simultaneously with the infection (FIG. 14).

In our hRSV Balb/C mouse model (experiment 4), no weight loss or clinical signs are usually observed following infection. Yet, infected mice treated with the PAR-1 antagonist showed significantly reduced viral titers on day 5 post infection compared to untreated and PAR-1 agonist treated mice (FIG. 15).

### CONCLUSIONS

A three-day prophylactic treatment of hMPV-infected BALB/c mice with the PAR-1 agonist resulted in a dose-dependent increase in disease severity compared to untreated, hMPV-infected mice, demonstrated by an increase in weight loss, symptoms and mortality. On day 5 post infection, this increase in disease severity was not accompanied by an increase in pulmonary viral titers, but there were dose-dependent increases in IL-6, IL-12(p40) and MCP-1 suggesting that PAR-1 activation has a detrimental effect on the immune response and immune cell recruitment rather than on direct viral replication in the lungs.

In contrast, a three-day prophylactic treatment of hMPV-infected BALB/c mice with the PAR-1 antagonist resulted in a dose-dependent reduction in disease severity compared to untreated, hMPV-infected mice. PAR-1 antagonist-treated mice started losing weight at a later time-point and lost less weight than untreated mice. A significant reduction in pulmonary viral titers was observed for PAR-1 antagonist treated mice on day 5 post infection, but a 3-day prophylactic treatment may not have been sufficient to result in a decrease in cytokine expression on day 5 post infection. Therefore the animal protocol was repeated, this time giving a five-day prophylactic treatment to hMPV-infected mice.

After a five-day prophylactic treatment of hMPV-infected mice with the PAR-1 agonist or a control peptide, no significant difference in disease severity (weight loss, symptoms and mortality) was observed. No significant difference in pulmonary viral titers and pulmonary inflammation, between PAR-1 agonist-treated and untreated mice was observed on day 5 post infection. It has been suggested that PAR-1 activation at a later time-point may have a paradoxical effect; early PAR-1 activation has been shown to have a detrimental effect on sepsis, while late PAR-1 activation appears to have a beneficial effect (Kaneider et al, Nature Immunology 8, 1303-1312 (2007)) Importantly, the five-day prophylactic treatment of hMPV-infected mice with the PAR-1 antagonist completely protected mice from any signs of illness (weight loss, symptoms and mortality); in fact, no significant difference in weight loss was observed between hMPV-infected PAR-1 antagonist treated mice and uninfected, untreated mice. This was confirmed by the evaluation of the pulmonary expression of inflammatory cytokines/chemokines and also by histopathological analysis of lungs harvested on day 5 post hMPV infection. Viral replication in the lungs of infected, PAR-1 antagonist-treated mice was significantly reduced compared to all other groups. This could suggest an involvement of the immune system in hMPV pathogenesis. Finally, uninfected mice were also given a 5-day prophylactic treatment with either the PAR-1 agonist, PAR-1 antagonist or the control peptide, to verify the absence of any toxic and non specific effects of the compounds.

The protective effect of a 5-day treatment with the PAR-1 antagonist was still present when the treatment was delayed 24 h post infection. However, dose optimisation or increasing the duration of the PAR-1 antagonist treatment might result in a greater clinical benefit even in a post-infection treatment setting.

Even though the Balb/c mouse model used here does not usually result in weight loss or clinical signs in hRSV-infected mice, a 5-day treatment with the PAR-1 antagonist significantly reduced pulmonary viral titers on day 5 post infection, suggesting that the effects observed in hMPV-infected mice can be extended to other members of the Paramyxoviridae family.

In summary, these animal experiments confirm the important role of PAR-1 receptors on hMPV and hRSV infections. When administered at the same time of or shortly after viral infection, PAR-1 agonists seem to have detrimental effects on clinical, immunological and histopathological endpoints whereas PAR-1 antagonists have a beneficial effect by improving weight, clinical symptoms while reducing lung viral titers and inflammation.

Headings are included herein for reference and to aid in locating certain sections. These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a compound” includes one or more of such compounds, and reference to “the method” includes reference to equivalent steps and methods known to those of ordinary skill in the art that could be modified or substituted for the methods described herein.

Unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A method for preventing or treating a Paramyxoviridae infection in a subject, comprising administering to said subject a Protease-Activated Receptor-1 (PAR1) inhibitor.

2. The method according to claim 1, wherein said PAR1 inhibitor is selected from the group consisting of peptides, peptides mimetic, chemically synthesized organic molecules, aptamers, siRNAs, pepducins, polynucleotides and antibodies.

3. The method according to claim 1, wherein said inhibitor is a PAR1 antagonist.

4. The method according to claim 3, wherein said PAR1 antagonist is selected from the group consisting of N3-cyclopropyl-7-{[4(1-methylethyl)phenyl]methyl)-7H-pyrrolo[3,2-f]quinazoline-1,3-diamine} (SCH-79797), Vorapaxar (SCH-530348), Atopaxar (E5555) and SCH-602539.

5. The method according to claim 1, wherein said subject is a mammal or an avian.

6. The method according to claim 1, wherein said subject is a human.

7. The method according to claim 1, wherein said Paramyxoviridae infection is an infection by a virus of the Subfamily Pneumovirinae.

8. The method according to claim 7, wherein the virus of the Subfamily Pneumovirinae is a virus from the genus pneumovirus or from the genus metapneumovirus.

9. The method according to claim 7, wherein the virus of the Subfamily Pneumovirinae is a human respiratory syncytial virus (hRSV) or a human metapneumovirus (hMPV).

10. The method of claim 1, wherein said preventing or treating comprises reducing morbidity and/or reducing mortality associated with said Paramyxoviridae infection.

11. The method of claim 1, wherein said preventing or treating comprises reducing inflammation, preventing weight loss, increasing survival and/or reducing viral titers associated with said Paramyxoviridae infection.

12. The method of claim 1, wherein said inhibitor is administered prophylactically before infection or within two days after infection or within two days of appearance of symptoms of infection.

13. A method for the prevention or treatment of Pneumovirinae infection in a human subject, comprising administering to said subject a Protease-Activated Receptor-1 (PAR1) inhibitor before infection, within two days after infection or within two days of appearance of symptoms of infection.

14.-25. (canceled)

26. A pharmaceutical composition for the prevention or treatment of a Paramyxoviridae infection in a subject, said composition comprising a Protease-Activated Receptor-1 (PAR1) inhibitor and a pharmaceutically acceptable carrier.

27. The pharmaceutical composition according to claim 26, wherein said composition further comprises a neuraminidase inhibitor.

28. The pharmaceutical composition according to claim 27, wherein said neuraminidase inhibitor is selected form the group consisting of: Oseltamivir, Zanamivir, Laninamivir and Peramivir.

29. The pharmaceutical composition according to claim 26, wherein said composition further comprises a compound selected from the group consisting of: ribavirin, peginterferon alfa-2b, peginterferon alfa-2a, antibiotics, and anti-inflammatory compounds.

30. An antiviral composition comprising a Protease-Activated Receptor-1 (PAR1) inhibitor in combination with a neuraminidase inhibitor.

31. The method of claim 13, wherein the Pneumovirinae infection is a human respiratory syncytial virus (hRSV) infection or a human metapneumovirus (hMPV) infection.

32. The method of claim 13, wherein said PAR1 inhibitor is selected from the group consisting of N3-cyclopropyl-7-{[4(1-methylethyl)phenyl]methyl)-7H-pyrrolo[3,2-f]quinazoline-1,3-diamine} (SCH-79797), Vorapaxar (SCH-530348), Atopaxar (E5555) and SCH-602539.
